(19) United States
(12) Patent Application Publication
(10) Pub. No.: US 2024/0285643 A1
(43) Pub. Date: Aug. 29, 2024

(54) TREATING INFLUENZA USING SUBSTITUTED POLYCYCLIC PYRIDONE DERIVATIVES AND PRODRUGS THEREOF IN A SUBJECT HAVING INFLUENZA AND A SEVERE INFLUENZA CONDITION

(71) Applicants: Shionogi & Co., Ltd., Osaka (JP); F. HOFFMANN-LA ROCHE AG, Basel (CH)

(72) Inventors: Takeki UEHARA, Osaka (JP); Toru ISHIBASHI, Osaka (JP); Takao SHISHIDO, Osaka (JP); Keita FUKAO, Osaka (JP); Motoyasu OONISHI, Osaka (JP); Barry CLINCH, Welwyn Garden City (GB); Jaspinder RANDHAWA, Welwyn Garden City (GB)

(21) Appl. No.: 18/602,656

(22) Filed: Mar. 12, 2024

Related U.S. Application Data
(63) Continuation of application No. 17/818,294, filed on Aug. 8, 2022.
Application No. 17/818,294 is a continuation of application No. 16/825,263, filed on Mar. 20, 2020, now abandoned.

(30) Foreign Application Priority Data
Mar. 22, 2019 (WO) PCT/IB2019/052349
Sep. 24, 2019 (WO) PCT/IB2019/058102

(51) Int. Cl. A61K 31/5383; A61K 31/215; A61P 31/16

(52) U.S. Cl. CPC A61K 31/5383; A61K 31/215; A61P 31/16

(57) ABSTRACT

A method for treating an influenza virus infection is described. The disclosed method generally involves administering an effective amount of a compound (A), for example baloxavir marboxil, and a compound (B), for example a neuraminidase inhibor, to a subject that (1) has an influenza virus infection, (2) has been symptomatic of the influenza virus infection for no more than 96 hours, and (3) further has at least one severe influenza condition selected from the following: (a) being hospitalized due to severe influenza virus infection, (b) requiring an extension of hospitalization because of the influenza virus infection during the hospitalization, (c) having a National Early Warning Score 2 of four or more, (d) being on support for respiration, and (e) having at least one complication attributable to the influenza virus infection that necessitates hospitalization.

### CROSS-REFERENCE TO RELATED APPLICATIONS

This application is a Continuation application of U.S. patent application Ser. No. 17/818,294, filed on Aug. 8, 2022, which is a Continuation application of U.S. patent application Ser. No. 16/825,263, filed on Mar. 20, 2020, which claims benefit of priority to International Patent Application No. PCT/IB2019/058102, filed on Sep. 24, 2019 and International Patent Application No. PCT/IB2019/052349, filed on Mar. 22, 2019, all of which are incorporated herein by reference in their entirety.

### STATEMENT REGARDING FEDERALLY SPONSORED RESEARCH OR DEVELOPMENT

This application contains an invention made with Government support under Agreement HHSO100201800036C, awarded by HHS. The Government has certain rights in the invention.

### FIELD

The present disclosure relates generally to treating an influenza virus infection in a subject having an influenza virus infection and a severe influenza condition, using a substituted polycyclic pyridone derivative having cap-dependent endonuclease inhibitory activity, a prodrug thereof, and a pharmaceutical composition including the same.

### BACKGROUND

Influenza is an acute respiratory infectious disease caused by a virus of the orthomyxovirus family. Two forms, such as influenza type A and type B, are known to infect humans. These viruses cause an acute febrile infection of a respiratory tract after an incubation period from 1 to 4 days, characterized by a sudden onset of fever, cough, fatigue, headache, and/or myalgia. Annual influenza epidemics are thought to result in between 3 and 5 million cases as severe ill cases, and between 250,000 and 500,000 deaths, every year around the world (WHO 2017).

Although in general, influenza is a disease that can be cured through natural defenses in healthy adults, the disease can be associated with substantial morbidity and occasional mortality in children, elderly, and immunocompromised patients (Non-Patent Document 1). Hospitalization due to severe influenza condition may result in high mortality (4%-8%), intensive care unit (ICU) admission (5%-17%), and prolonged hospital stays between 5 and 9 days. During a pandemic season, the outcomes may be more serious, such that up to 34% of patients require ICU care and that a mortality rate is as high as 15% (Non-Patent Document 2).

The following anti-influenza virus drugs are currently available for treatment of acute, uncomplicated influenza virus infection in various countries: the M2 ion channel inhibitors (e.g., amantadine and rimantadine), the RNA polymerase inhibitor (e.g., favipiravir), and the neuraminidase (NA) inhibitors (i.e., oseltamivir, zanamivir, and peramivir). In many cases of seasonal influenza virus infection, the viruses are resistant to amantadine and rimantadine, hence their use in clinical practice is limited. Oral formulations of the NA inhibitor (NAI) need to be administered for 5 days, potentially resulting in poor patient compliance and convenience, while inhalation formulations can only be used in patients who are able to inhale the drug. Due to these unmet-medical-needs, new antiviral influenza drugs that can be easily and less frequently administered have been sought, particularly for patients who are severely ill and possibly intubated.

Several new influenza antivirals that target different protein subunits of an influenza polymerase complex are under clinical studies (Non-Patent Document 3). Baloxavir marboxil (BXM) is a small-molecule prodrug of baloxavir, which has an antiviral activity against influenza type A and type B viruses, including those resistant to current antivirals (Non-Patent Document 4). BXM was recently approved in the US, for treatment of uncomplicated influenza virus infection in otherwise healthy individuals ≥12 years old. BXM provided more rapid reductions in infectious virus titers than placebo or oseltamivir (Non-Patent Document 5).

There are no approved drugs for marketing for the treatment of influenza virus infection in hospitalized patients, since there have been no evidence showing such effectiveness in, randomized-controlled clinical trials assessing the effectiveness of NAI treatment against placebo. Despite luck of the evidence, the NAIs have been widely used as the mainstay of treatment for hospitalized patients, and evidence shows a potential reduction in mortality in hospitalized patients treated with NAIs, especially if the treatment is initiated in an early stage, for esample within 48 hours from onset of at least one symptom of influenza virus infection (Non-Patent Document 6).

Patent Document 1-6 describe BXM and/or compounds having similar structures to substituted polycyclic pyridone derivatives.
Patent Document 1: WO2010/147068
Patent Document 2: WO2012/039414
Patent Document 3: WO2016/175224
Patent Document 4: WO2017/104691
Patent Document 5: WO2017/221869
Patent Document 6: WO2018/030463
Non-Patent Document 1: Paules C, Subbarao K. Influenza. Lancet 2017, 390, 697-708.
Non-Patent Document 2: Lee N, Ison M G. Diagnosis, management and outcomes of adults hospitalized with influenza. Antivir Ther 2012; 17(1 Pt B): 143-57.
Non-Patent Document 3: McKimm-Breschkin J L, Jiang S, Hui D S, Beigel J H, Govorkova E A, Lee N. Prevention and treatment of respiratory viral infections: Presentations on antivirals, traditional therapies and host-directed interventions at the 5th ISIRV Antiviral Group conference. Antiviral Res 2018; 149:118-42.
Non-Patent Document 4: Uehara T, Shishido T, Ishibashi T, et al. S-033188, a Small Molecule Inhibitor of Cap-dependent Endonuclease of Influenza type A and type B Virus, Leads to Rapid and Profound Viral Load Reduction. Options IX for the Control of Influenza Chicago, Illinois 2016.
Non-Patent Document 5: Hayden F G, Sugaya N, Hirotsu N, et al. Baloxavir Marboxil for Uncomplicated Influenza in Adults and Adolescents. N Engl J Med 2018; 379:913-23.
Non-Patent Document 6: Muthuri S G, Venkatesan S, Myles P R, et al. Effectiveness of neuraminidase inhibitors in reducing mortality in patients admitted to hospital with influenza A H1N1pdm09 virus infection: a meta-analysis of individual participant data. Lancet Respir Med 2014, 2, 395-404.

### SUMMARY

A method for treating an influenza virus infection is described. The disclosed method generally involves administering a combination of an effective amount of a compound (A) and an effective amount of a compound (B) to a subject, wherein the subject (1) has an influenza virus infection, (2) has been symptomatic of the influenza virus infection for no more than 96 hours, and (3) further has at least one severe condition of influenza virus infection (severe influenza condition) that is one or more of: (a) being hospitalized due to the influenza virus infection, (b) requiring an extension of hospitalization because of acquiring the influenza virus infection during the hospitalization, (c) having a National Early Warning Score 2 of four or more, (d) being on support for respiration, and (e) having at least one complication attributable to the influenza virus infection that necessitates hospitalization.

In one example, the compound (A) has one of the following formulae:

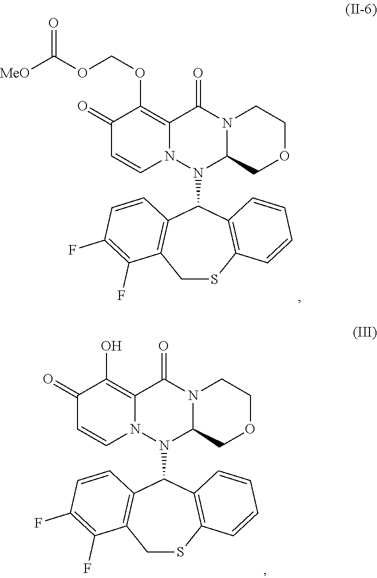 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof.

In one example, the compound (B) is at least one neuraminidase inhibitor.

In one example, the neuraminidase inhibitor is one or more of oseltamivir, zanamivir and peramivir, a pharmaceutically acceptable salt thereof, and a pharmaceutically acceptable solvate thereof. In one example, the neuraminidase inhibitor is oseltamivir, a pharmaceutically acceptable salt thereof, or a pharmaceutically acceptable solvate thereof.

In one example, the onset of the influenza virus infection is the time point when the subject newly has at least one symptom that is at least one systemic symptom and/or at least one respiratory symptom.

In one example, the at least one systemic symptom includes one or more of headache, feverishness, chills, muscular pain, joint pain, and fatigue.

In one example, the at least one respiratory symptom includes one or more of coughing, sore throat, and nasal congestion.

In one example, a condition that necessitates support for respiration is at least one of a ventilator and an inhalation of oxygen from non-atmospheric oxygen supply, and an oxygen concentrator which concentrates the atmospheric oxygen.

In one example, at least one complication attributable to the influenza virus infection is present. In one example, the complication attributable to the influenza virus infection is one or more of inflammation of heart, brain, or muscle tissues, and muti-organ failure. In one example, the complication attributable to the influenza virus infection is one or more of pneumonia, central nervous system involvement, myositis, rhabdomyolysis, encephalitis, encephalopathy, severe dehydration myocarditis, pericarditis, otitis media, sinusitis, exacerbation of ischemic heart disease, sepsis, acute lung injury, or acute respiratory distress syndrome, and acute exacerbation of chronic kidney disease or respiratory diseases, for example asthma or chronic obstructive pulmonary disease.

In one example, the compound (A) is administered in an amount from about 40 mg to about 80 mg. In one example, the compound (A) can be administered as a weight-based dose. In one example, about 40 mg is administered to a subject weighing about 40 kg to less than about 80 kg. In one example, about 80 mg is administered to a subject weighing at least 80 kg.

In one example, the subject is a patient that is younger than 1 year. In this instance:
(a) if the patient is younger than 3 months, then the effective amount is 0.8-1.2 mg/kg body weight, preferably about 1 mg/kg body weight; or
(b) if the patient is 3 months or older but younger than 12 months, then the effective amount is 1.8-2.2 mg/kg body weight, preferably about 2 mg/kg body weight.

In one example, the subject is a patient that is 1 year or older but younger than 12 years. In this instance:
(a) if the patient has a body weight of less than 20 kg, then the effective amount is 1.8-2.2 mg/kg body weight, preferably about 2 mg/kg body weight; or
(b) if the patient has a body weight of 20 kg or more, then the effective amount is 35-45 mg, preferably about 40 mg.

In one example, the compound (A) is administered on the first day of onset of at least one symptom of an influenza virus infection, and again three days after the first day of administration upon onse of at least one symptom of an influenza virus infection. In one example, the compound (A) is administered on the first day of onset of at least one symptom of an influenza virus infection, and again three days and six days after the first day of administration upon onset of at least one symptom of an influenza virus infection.

In one example, the compound (A) is administered two times or three times in total.

In one example, the compound (A) is administered at day 1 or day 4 after the onset of the influenza virus infection. The term “Day 1” indicates the first day of administration. The term “Day 4” indicates the fourth day as counted from the first day of administration.

In one example, the compound (A) is further administered at day 7, i.e., the seventh day from the first day of administration.

In one example, the effective amount of the compound (B) is in a range from about 0.1 to about 6000 mg as an active compound. In another example, the effective amount of the compound (B) is in a range from about 0.1 to about 1500 mg as an active compound.

In one example, the compound (B) is oseltamivir phosphate, and the effective amount administered is about 75 mg twice daily for five days as an active compound.

In one example, the compound (B) is zanamivir hydrate, and the effective amount administered is 10 mg twice daily for five days as an active compound.

In one example, the compound (B) is peramivir hydrate. For adults, the effective amount administered is 600 mg once daily for five days as an active compound. For adolescents, the effective amount is 10 mg/kg up to a maximum of 600 mg, once daily for five days as an active compound.

In one example, the compound (B) is administered at one time. In one example, the compound (B) is administed once daily for up to ten days after the onset of the influenza virus infection.

In one example, the amount of the compound (A) and the amount of the compound (B) administered are effective such that a time to show clinical improvement in the subject is statistically significant as compared to a treated subject with the compound (B).

In one example, the amount of the compound (A) and the amount of the compound (B) administered are effective such that a time to show clinical improvement in the subject is statistically significant as compared to a non-treated subject. A non-treated subject is a subject that has not been administered with the compound (A) and the compound (B).

In one example, a p-value indicating the statistical significance of the time to show clinical improvement is less than 0.05, alternately less than 0.005.

In one example, the time to clinical improvement is a time to hospital discharge or a time until a National Early Warning Score 2 of two or less is maintained for at least 24 hours.

In one example, each of the compound (A) and the compound (B) is administered through a route individually selected from the group consisting of orally or parenterally.

In one example, the compound (A) is administered at day 7 after the onset of the influenza virus infection in the case that the subject does not show an improvement in at least one condition selected from the group consisting of (i) continuous use of a ventilator, (ii) continuous fever, (iii) severe immune deficiency, and (iv) any complication attributable to the influenza virus infection.

### ASPECTS

1. A method for treating an influenza virus infection, comprising:
   administering a combination of an effective amount of a compound (A) and an effective amount of a compound (B) to a subject,
   wherein the subject:
   (1) has an influenza virus infection,
   (2) has been symptomatic of the influenza virus infection for no more than 96 hours, and
   (3) further has at least one severe influenza condition selected from the group consisting of:
      (a) being hospitalized due to influenza virus infection,
      (b) requiring an extension of hospitalization because of acquiring the influenza virus infection during the hospitalization,
      (c) having a National Early Warning Score 2 of four or more,
      (d) being on support for respiration, and
      (e) having at least one complication attributable to the influenza virus infection that necessitates hospitalization,
   wherein the compound (A) has one of the following formulae:

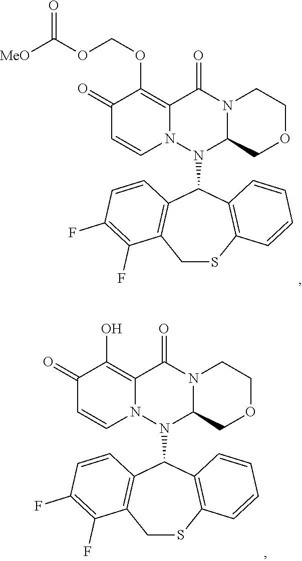 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof, and
wherein the compound (B) is at least one neuraminidase inhibitor.
2. The method of aspect 1, wherein the at least one neuraminidase inhibitor is at least one compound selected from the group consisting of oseltamivir, zanamivir, peramivir, a pharmaceutically acceptable salt thereof, and a pharmaceutically acceptable solvate thereof.
3. The method of aspect 1 or 2, wherein the at least one neuraminidase inhibitor is oseltamivir, a pharmaceutically acceptable salt thereof, or a pharmaceutically acceptable solvate thereof.
4. The method of any one of aspects 1 to 3, wherein the at least one symptom is at least one systemic symptom selected from the group consisting of headache, feverishness, chills, muscular pain, joint pain, and fatigue.
5. The method of aspect 4, wherein the at least one symptom is at least one respiratory symptom selected from the group consisting of coughing, sore throat, and nasal congestion.
6. The method of any one of aspects 1 to 5, wherein the severe influenza condition is being on support for respiration, and the support for respiration is selected from the group consisting of a ventilator, inhalation of oxygen from non-atmospheric oxygen supply, and an oxygen concentrator that concentrates atmospheric oxygen.
7. The method of any one of aspects 1 to 6, wherein the severe influenza condition is having the at least one complication attributable to the influenza virus infection selected from the group consisting of pneumonia, central nervous system involvement, myositis, rhabdomyolysis, severe dehydration myocarditis, pericarditis, exacerbation of ischemic heart disease, and acute exacerbation of chronic kidney disease, asthma, and chronic obstructive pulmonary disease.
8. The method of any one of aspects 1 to 7, wherein the compound (A) is administered in an amount from about 80 mg to about 240 mg.
9. The method of any one of aspects 1 to 8, wherein compound (A) is administered two times or three times in total.
10. The method of any one of aspects 1 to 9, wherein the compound (A) is administered at day 1 or day 4 after the onset of the influenza virus infection.
11. The method of any one of aspects 1 to 10, wherein the compound (A) is further administered at day 7 after the onset of the influenza virus infection.
12. The method of any one of aspects 1 to 11, wherein the effective amount of the compound (B) is in a range from about 0.1 mg to about 6000 mg as an active compound.
13. The method of any one of aspects 1 to 12, wherein the compound (B) is administered at one time or daily for up to five days after the onset of the influenza virus infection.
14. The method of any one of aspects 1 to 12, wherein the compound (B) is administered at one time or daily for up to ten days after the onset of the influenza virus infection.
15. The method of any one of aspects 1 to 14, wherein the amounts of the compound (A) and the compound (B) administered are effective such that a time to show clinical improvement in the subject is reduced compared to the time of a treated subject only with the compound (B).
16. The method of any one of aspects 1 to 14, wherein the amounts of the compound (A) and the compound (B) administered are effective such that a time to show clinical improvement in the subject is reduced compared to the time of a non-treated subject.
17. The method of aspect 15, wherein the amounts administered are effective such that the time to show clinical improvement in the subject is statistically significant as compared to the time of a treated subject only with the compound (B).
18. The method of aspect 17, wherein a p-value indicating the statistical significance of the time to show the clinical improvement is less than 0.05.
19. The method of aspect 15 or 16, wherein the time to show clinical improvement is a time to hospital discharge or a time until a National Early Warning Score 2 of two or less is maintained for at least 24 hours.
20. The method of any one of aspects 1 to 19, wherein each of the compound (A) and the compound (B) is administered through a route individually selected from the group consisting of orally or parenterally.
21. The method of any one of aspects 1 to 20, wherein the compound (A) is administered at day 7 after the onset of the influenza virus infection in the case that the subject does not show an improvement in at least one condition selected from the group consisting of (i) continuous use of a ventilator, (ii) continuous fever, (iii) severe immune deficiency, and (iv) any complication attributable to the influenza virus infection.
22. A method for treating an influenza virus infection, comprising: reading a dosage instruction on a package insert or in a package for a pharmaceutical formulation comprising a compound (A) having one of the following formulae:

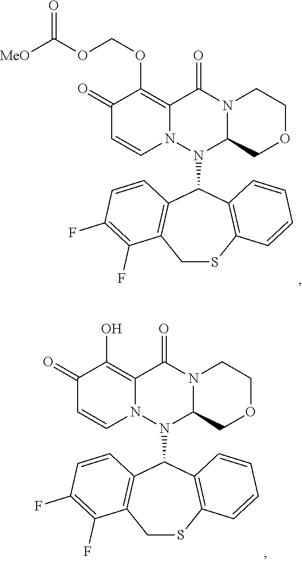 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof, and a compound (B) that is at least one neuraminidase inhibitor; and
   administering the pharmaceutical formulation to a subject that:
   (1) has an influenza virus infection,
   (2) has been symptomatic of the influenza virus infection for no more than 96 hours, and
   (3) further has at least one severe influenza condition selected from the group consisting of:
      (a) being hospitalized due to influenza virus infection,
      (b) requiring an extension of hospitalization because of acquiring the influenza virus infection during the hospitalization,
      (c) having a National Early Warning Score 2 of four or more,
      (d) being on support for respiration, and
      (e) having at least one complication attributable to the influenza virus infection that necessitates hospitalization.
23. A use of a compound (A) having one of the following formulae:

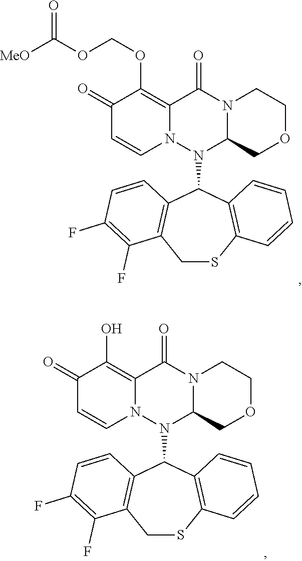 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof, and a compound (B) that is at least one neuraminidase inhibitor, for preparation of a medicament for treating a subject that:
   (1) has an influenza virus infection,
   (2) has been symptomatic of the influenza virus infection for no more than 96 hours,
   (3) further has at least one severe influenza condition selected from the group consisting of:
      (a) being hospitalized due to influenza virus infection,
      (b) requiring an extension of hospitalization because of acquiring the influenza virus infection during the hospitalization,
      (c) having a National Early Warning Score 2 of four or more,
      (d) being on support for respiration, and
      (e) having at least one complication attributable to the influenza virus infection that necessitates hospitalization,
   wherein the treatment includes administering an effective amount of the compound (A) and an effective amount of the compound (B) to the subject.
24. A pharmaceutical composition useful for treating a subject that:
   (1) has an influenza virus infection,
   (2) has been symptomatic of the influenza virus infection for no more than 96 hours, and
   (3) further has at least one severe influenza condition selected from the group consisting of:
      (a) being hospitalized due to influenza virus infection,
      (b) requiring an extension of hospitalization because of acquiring the influenza virus infection during the hospitalization,
      (c) having a National Early Warning Score 2 of four or more,
      (d) being on support for respiration, and
      (e) having at least one complication attributable to the influenza virus infection that necessitates hospitalization
   wherein the pharmaceutical composition comprises the compound (A) and the compound (B),
   the treatment comprises administering an effective amount of a compound (A) and an effective amount of a compound (B) to the subject, and
   the compound (A) has one of the following formulae:

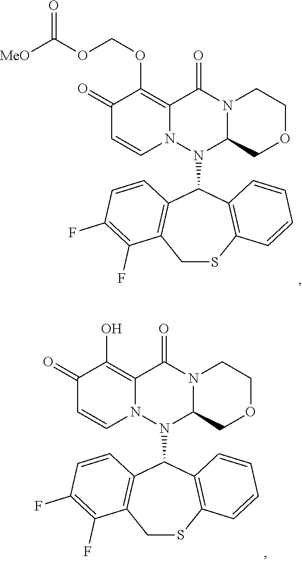 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof, and wherein the compound (B) is at least one neuraminidase inhibitor.
25. A package, comprising a pharmaceutical formulation comprising a compound (A) having one of the following formulae:

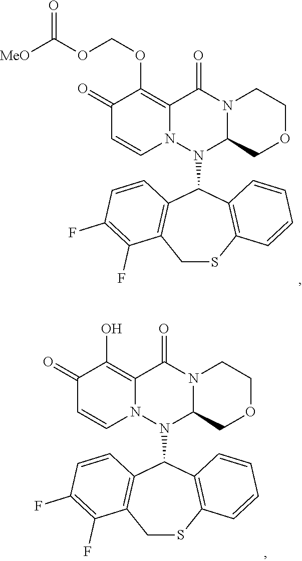 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof, and a compound (B) that is at least one neuraminidase inhibitor; and a dosage instruction on a package insert or in a package for administering the pharmaceutical formulation to a subject that:
   (1) has an influenza virus infection,
   (2) has been symptomatic of the influenza virus infection for no more than 96 hours, and
   (3) further has at least one severe influenza condition selected from the group consisting of:
      (a) being hospitalized due to influenza virus infection,
      (b) requiring an extension of hospitalization because of acquiring the influenza virus infection during the hospitalization,
      (c) having a National Early Warning Score 2 of four or more,
      (d) being on support for respiration, and
      (e) having at least one complication attributable to the influenza virus infection that necessitates hospitalization.

### BRIEF DESCRIPTION OF THE DRAWINGS

FIG. 1 is a graph showing the experimental results of measuring the plasma concentration of Compound (III) (baloxavir or “BXA”), after oral administration of prodrug Compound (II-6) (baloxavir marboxyl or “BXM”), to rats under non-fasting conditions.

FIG. 2 is a table showing the experimental results of measuring the plasma concentration of Compound (II-6) (BXM), after oral administration, to rats under non-fasting conditions.

FIGS. 3A and 3B are graphs of experimental results showing the therapeutic efficacy of 2-day delayed administration of Compound (III) (BXA) against lethal co-infection with influenza type A virus (inoculation: day 0) and Streptococcus pneumoniae (inoculation: day 2) in mice. Mice were monitored daily for survival and body weight through 15 days post infection. Significant difference in survival time was observed in groups treated with BXA in comparison with vehicle treated group (**, P<0.01). The survival time in groups that treated with oseltamivir phosphate (“OSP”) was significantly prolonged compared to that in vehicle treated group (*, P<0.05). The survival time of the group that received BXA or BXA+OSP was significantly prolonged compared to that of the group treated with OSP (^{†}, P<0.05; ^{‡}, P<0.01).

FIGS. 4A and 4B are graphs of experimental results showing the therapeutic efficacy of 3-day delayed administration of Compound (III) against lethal co-infection with influenza type A virus (inoculation: day 0) and Streptococcus pneumoniae (inoculation: day 2) in mice. Mice were monitored daily for survival and body weight through 15 days post infection. When the treatment was delayed until 3 days post virus infection, a significant difference in survival time was observed in groups treated with BXA+OSP in comparison with the vehicle treated group (*, P<0.05).

FIGS. 5A and 5B are graphs of experimental results showing the therapeutic efficacy of 4-day delayed administration of Compound (III) against lethal co-infection with influenza type A virus (inoculation: day 0) and Streptococcus pneumoniae (inoculation: day 2) in mice. Mice were monitored daily for survival and body weight through 15 days post infection.

FIG. 6 is a table showing a summary of the National Early Warning Score 2 (NEWS).

FIG. 7 is an overview of the design of the clinical trial from Test Example 3.

### DETAILED DESCRIPTION

A method for treating an influenza virus infection is described. The disclosed method generally involves administering a combination of an effective amount of a compound (A) and an effective amount of a compound (B) to a subject, wherein the subject (1) has an influenza virus infection, (2) is at 96 hours or less since the onset of the influenza virus infection when the combination is initially administered, and (3) further has at least one severe influenza condition.

Generally, the compound (A) that can be used in the disclosed method is described as follows.
(1) A compound represented by the following formula (I):

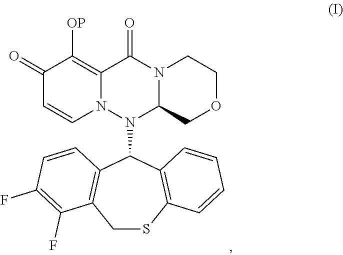

wherein P is hydrogen or a group to form a prodrug, or its pharmaceutically acceptable salt.
(2) The compound according to (1), or its pharmaceutically acceptable salt, wherein P is a group to form a prodrug selected from the group consisting of:

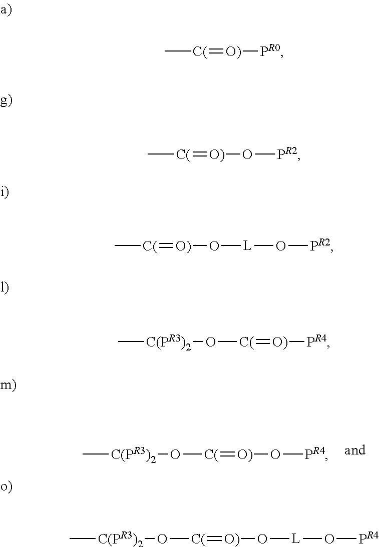

wherein L is straight or branched lower alkylene;
P^{R0} is alkyl;
P^{R2} is alkyl;
P^{R3} is each independently hydrogen; and
P^{R4} is alkyl.

In one example, the compound (A) that can be used in the disclosed method has a formula:

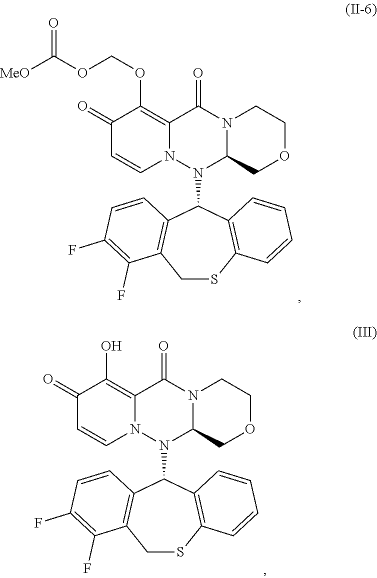 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt thereof.

The meaning of various terms used in the present description is explained below. Each term is used in a unified sense, and is used in the same sense when used alone, or when used in combination of other term.

“Prodrug” in the present description refers to a compound represented by formula (II) in the following reaction formula:

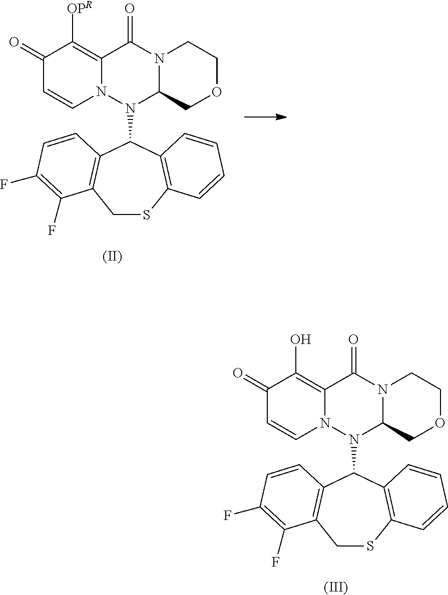

wherein P^{R} is a group to form a prodrug, or its pharmaceutically acceptable salt.

“Group to form a prodrug” in the present description refers to a “P^{R}” group in the formula (II), in the following reaction formula:

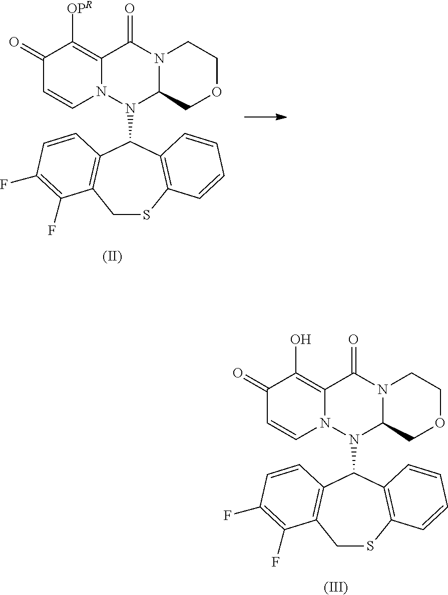

wherein P^{R} is selected from the group consisting of:

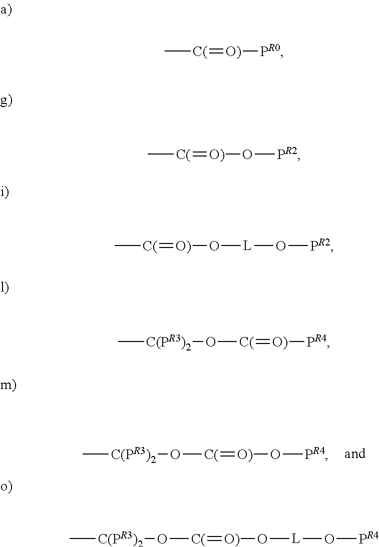

wherein L is straight or branched lower alkylene;
P^{R0} is alkyl;
P^{R2} is alkyl;
P^{R3} is each independently hydrogen; and
P^{R4} is alkyl.

“Converted into a prodrug” in the present description means that, as shown in the following reaction formula:

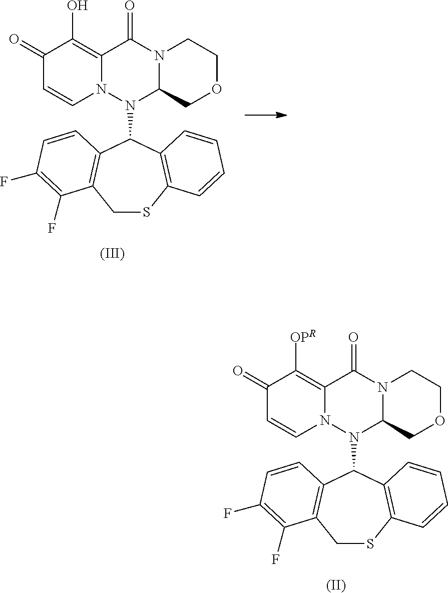

wherein P^{R} is a group to form a prodrug,
a hydroxy group in the formula (III) or its pharmaceutically acceptable salt is converted into —OP^{R} group.

“Parent compound” in the present description means a compound to be a source before synthesizing the “prodrug” and/or a compound released from the “prodrug” by the reaction by enzymes, a gastric acid, and the like under physiological conditions in vivo, and specifically means a compound shown by the formula (III), or pharmaceutically acceptable salt thereof or a solvate thereof.

Examples of one embodiment of compounds that can be used for the compound (A) in the present description include compounds that are described in the PCT/JP2016/063139 application published as WO 2016/175224A1.

The term “alkyl” includes a C1 to C15, alternatively a C1 to C10, alternatively a C1 to C6, alternatively a C1 to C4, linear or branched hydrocarbon group. Examples of “alkyl” include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, n-decyl and the like.

One embodiment of “alkyl” is methyl, ethyl, n-propyl, isopropyl, n-butyl, isobutyl, sec-butyl, tert-butyl or n-pentyl. Another embodiment of “alkyl” is methyl, ethyl, n-propyl, isopropyl or tert-butyl.

The term “alkylene” includes a C1 to C15, alternately a C1 to C10, alternately a C1 to C6 and alternately a C1 to C4 linear or branched bivalent hydrocarbon group. Examples include methylene, ethylene, trimethylene, propylene, tetramethylene, pentamethylene, hexamethylene and the like.

One or more hydrogen, carbon and/or other atoms in the compounds used in the present invention may be replaced with isotopes of hydrogen, carbon and/or other atoms respectively. Examples of isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compounds used in the present invention include compounds replaced with these isotopes. The compounds replaced with the above isotopes are useful as medicines and include radiolabeled compounds of the compound used in the present invention. A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound used in the present invention can be prepared using well-known methods in the field of this invention. For example, a tritium-labeled compound used in the present invention can be prepared by introducing a tritium to a certain compound used in the present invention, through a catalytic dehalogenation reaction using a tritium. This method comprises reacting with an appropriately-halogenated precursor of the compound used in the present invention with tritium gas in the presence of an appropriate catalyst, such as Pd/C, and in the presence or absent of a base. The other appropriate methods of preparing a tritium-labeled compound can be found in “Isotopes in the Physical and Biomedical Sciences, Vol. 1, Labeled Compounds (Part A), Chapter 6 (1987)”. A ¹⁴C-labeled compound can be prepared by using a raw material having ¹⁴C.

The pharmaceutically acceptable salts of the compounds used in the present invention include, for example, salts with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Especially, salts with hydrochloric acid, sulfuric acid, phosphoric acid, tartaric acid, methanesulfonic acid and the like are included. These salts can be formed by the usual methods.

The compounds used in the present invention or its pharmaceutically acceptable salts may form solvates (e.g., hydrates or the like) and/or crystal polymorphs. The present invention encompasses those various solvates and crystal polymorphs. “Solvates” may be those wherein any numbers of solvent molecules (e.g., water molecules or the like) are coordinated with the compounds used in the present invention. When the compounds used in the present invention or its pharmaceutically acceptable salts are allowed to stand in the atmosphere, the compounds may absorb water, resulting in attachment of adsorbed water or formation of hydrates. Recrystallization of the compounds used in the present invention or its pharmaceutically acceptable salts may produce crystal polymorphs.

The group to form a prodrug is converted into OH group by action of drug-metabolizing enzymes, hydrolases, gastric acids, and/or enterobacteria, after in vivo administration (for example, oral administration).

Additionally, a prodrug shows improved bioavailability and/or AUC (area under the blood concentration curve) in in vivo administration compared to that of the Compound (III). A prodrug is efficiently absorbed into the body, e.g. from a stomach and/or intestines, after in vivo administration (for example, by oral administration), and then is converted into the Compound (III). Thus, the prodrug shows an effect of treating and/or preventing influenza virus infection higher than the Compound (III).

Examples of an embodiment of a particularly preferable substituent of the group to form a prodrug include following groups.

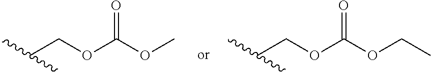

Other compounds that may be used for the compound (A) are described in in the PCT/JP2016/063139 application published as WO 2016/175224A1, all disclosures in which are herein incorporated by reference.

A general method for producing the compound (A) used in the present invention will be exemplified below. As to the extraction and purification, a treatment performed in a normal experiment of organic chemistry may be conducted.

Synthesis of the compound (A) used in the present invention can be carried out referring to the procedures known in the art.

As a raw material compound, commercially available compounds, compounds described in the present description, compounds described in the references cited in the present description, and other known compounds can be utilized.

When one wants to obtain a salt of the compound (A) used in the present invention, in the case where the compound (A) used in the present invention is obtained in a form of a salt, it may be purified as it is and, in the case where the compound (A) used in the present invention is obtained in a free form, a salt may be formed by a normal method by dissolving or suspending the compound (A) in a suitable organic solvent, and adding an acid or a base.

### (Preparation 1)

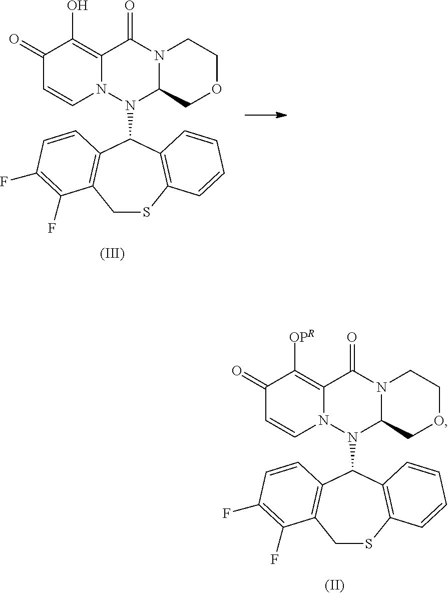

wherein P^{R} is a group to form a prodrug as set forth above.

Compound (II) can be obtained by a method including converting a hydroxyl group of Compound (III) into an ester group or ether group. The active agent (Compound (III)) can be used to prepare its prodrugs (i.e., compounds having the formula of Compound (II)).

For example, the method described in Protective Groups in Organic Synthesis, Theodora W Green (John Wiley & Sons), Prog. Med. 5: 2157-2161 (1985), and Supplied by The British Library—“The World's Knowledge”, etc. can be utilized. These references are herein incorporated by reference.

The compounds of formula III (Compound (III)) used in the present invention have a cap-dependent endonuclease inhibitory activity. Compound (III) and its prodrugs (i.e., Compound (II)) are useful as a therapeutic or preventive agent for influenza virus infection, particularly the compound of formula II-6 (Compound (II-6)) described below.

In general, for the purpose of treating the above-mentioned diseases in humans, the compounds used in the present invention may be administered orally as a powder, a granule, tablets, capsules, pills, a liquid and the like or parenterally as an injection, suppositories, a percutaneous drug, an inhalant and the like. The effective doses of the present compounds may be mixed with excipients suitable for the dosage form, such as fillers, binders, humectants, disintegrators, and lubricants, as appropriate, to form pharmaceutical preparations. For preparing an injection, sterilization is performed with a suitable carrier.

In general, the pharmaceutical compositions of the prodrugs (Compound (II)) and Compound (III) used in the present invention can be administered either orally or parenterally. For oral administration, commonly used dosage forms, such as tablets, granule, powder, and capsules, may be prepared according to conventional methods. For parenteral administration, any commonly used dosage form, such as an injection, may be suitably used. The compounds according to the present invention can be suitably used as oral preparations because of their high oral absorbability.

Generally, the dose of the prodrugs (Compound (II)) and Compound (III) depends on the condition of the disease, administration route, or age or weight of the patient. The usual oral dose for adults is in a range from 0.1 to 100 mg/kg per day, alternately in a range from 1 to 20 mg/kg per day. In some embodiments, patients weighing from 40 kg to less than 80 kg each receive a dose in a range from 80 mg to 120 mg. In other embodiments, patients weighing from 40 kg to less than 80 kg each receive a single dose of 40 mg. In other embodiments, patients weighing at least 80 kg receive a dose in a range from 160 to 240 mg. In other embodiments, patients weighing at least 80 kg receive a single dose of 80 mg. For patients younger than 1 year, the dose can be 0.8-1.2 mg/kg, alternately about 1.8-2.2 mg/kg, alternately 1 mg/kg, alternately 2 mg/kg. The compound can be administered orally, dermally, subcutaneously, intravenously, intraarterially, intramuscularly, intraperitoneally, transmucosally, via inhalation, transnasally, ophthalmically, via an inner ear and/or vaginally.

Generally, the compound (B) that can be used in the method of the present invention is at least one neuraminidase inhibitor. One neuraminidase inhibitor includes a pharmaceutically acceptable salt thereof and a pharmaceutically acceptable solvate thereof. In one example, the neuraminidase inhibitor can include one or more of oseltamivir, zanamivir, peramivir, laninamivir octanoate, a pharmaceutically acceptable salt thereof, and a pharmaceutically acceptable solvate thereof. In one example, the neuraminidase inhibitor is one or more of oseltamivir, zanamivir, peramivir, a pharmaceutically acceptable salt thereof, and a pharmaceutically acceptable solvate thereof. In one example, oseltamivir, a pharmaceutically acceptable salt thereof, or a pharmaceutically acceptable solvate thereof is oseltamivir phosphate. In one example, zanamivir, a pharmaceutically acceptable salt thereof, or a pharmaceutically acceptable solvate thereof is zanamivir hydrate. In one example, peramivir, a pharmaceutically acceptable salt thereof or a pharmaceutically acceptable solvate thereof is peramivir hydrate. In one example, laninamivir octanoate, a pharmaceutically acceptable salt thereof or a pharmaceutically acceptable solvate thereof is laninamivir octanoate hydrate.

In general, the compound (A) and the compound (B) used in the present invention can be used in combination with other drugs or the like (hereinafter referred to as combination drugs) to increase the activity of the compound, reduce the dose of the compound, or the like. In the case of treating influenza virus infection, the compound (A) and the compound (B) can be combined with or in a coupled formulation with RNA-dependent RNA polymerase inhibitor (e.g., Favipiravir); M2 protein inhibitor (e.g., Amantadine); PB2 Cap binding inhibitor (e.g., VX-787); anti-HA antibody (e.g., MHAA4549A); Immune agonists (e.g., Nitazoxanide) are also possible. In this case, the timing of administration for a compound used in the present invention and the combination drug is not limited. They can be administered to the subjects to be treated, at the same time or at different times. Furthermore, a compound used in the present invention and the combination drug can be administered as two or more formulations independently comprising each active ingredient or a single formulation comprising two or more active ingredients.

The dose for combination drugs may be appropriately selected in reference to the clinical dose. The compounding ratio of the compounds used in the present invention and co-administered drugs may be appropriately selected depending on the subject to be treated, administration route, disease to be treated, symptoms, combination of the drugs and the like. For administration in humans, for example, 1 part by weight of the compounds used in the present invention may be used in combination with 0.01 to 100 parts by weight of co-administered drugs.

### EXAMPLES

The present invention will be explained in more detail below by way of Examples, as well as Test Examples of the present invention, but the present invention is not limited to them.

The NMR analysis obtained in each reference example and example was carried out in 300 MHz, and was measured using DMSO-d₆, CDCl₃.

The term RT represents a retention time at LC/MS: liquid chromatography/mass spectrometry, and was measured under the following conditions.

### (Measurement Conditions)

(1) Column: ACQUITY UPLC (Registered trademark) BEH C18 (1.7 μm i.d.2.1×50 mm) (Waters)
   Flow rate: 0.8 mL/min
   UV detection wavelength: 254 nm
   Mobile phase: [A]: a 0.1% formic acid-containing aqueous solution, [B]: a 0.1% formic acid-containing acetonitrile solution
   Gradient: a linear gradient of 5% to 100% solvent [B] was carried out in 3.5 minutes, and 100% solvent [B] was kept for 0.5 minutes.

### Example 1

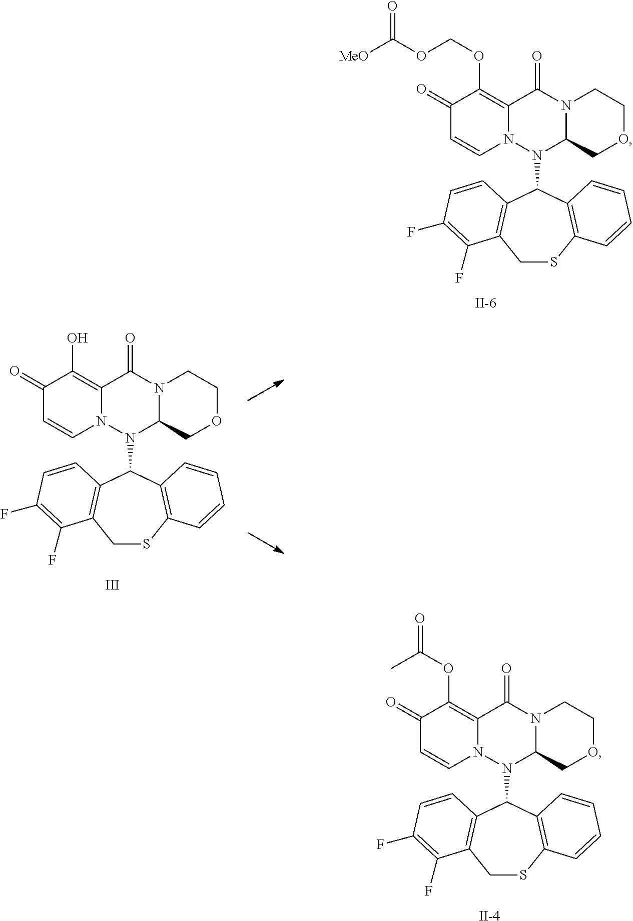

Compound (II-4) and (II-6) were synthesized from commercially available compounds according to the method described in WO2016/175224.

### Compound (II-6):

1H-NMR (DMSO-D6) δ: 2.91-2.98 (1H, m), 3.24-3.31 (1H, m), 3.44 (1H, t, J=10.4 Hz), 3.69 (1H, dd, J=11.5, 2.8 Hz), 3.73 (3H, s), 4.00 (1H, dd, J=10.8, 2.9 Hz), 4.06 (1H, d, J=14.3 Hz), 4.40 (1H, d, J=11.8 Hz), 4.45 (1H, dd, J=9.9, 2.9 Hz), 5.42 (1H, dd, J=14.4, 1.8 Hz), 5.67 (1H, d, J=6.5 Hz), 5.72-5.75 (3H, m), 6.83-6.87 (1H, m), 7.01 (1H, d, J=6.9 Hz), 7.09 (1H, dd, J=8.0, 1.1 Hz), 7.14-7.18 (1H, m), 7.23 (1H, d, J=7.8 Hz), 7.37-7.44 (2H, m).

### Compound (II-4):

1H-NMR (CDCl3) δ:2.46 (s, 3H), 2.88-2.99 (m, 1H), 3.35-3.50 (m, 1H), 3.60-3.65 (m, 1H), 3.75-3.83 (m, 1H), 3.90-4.00 (m, 1H), 4.05 (d, J=14.0 Hz, 1H), 4.52-4.57 (m, 1H), 4.60-4.70 (m, 1H), 5.24-5.34 (m, 1H), 5.35 (s, 1H), 5.88 (d, J=7.6 Hz, 1H), 6.85-6.82 (m, 1H), 6.90-7.05 (m, 2H), 7.06-7.20 (m, 4H)

$$LC / MS ( ESI ) : m / z = {526.2 [ M + H ]}^{+} , R T = 1.87 min , method \qquad ( 1 )$$

The following example compounds in Table 1 were synthesized from commercially available compounds according to the above examples.

TABLE 1

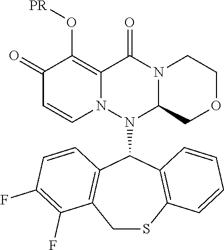

| No. | PR | data | comment |
|---|---|---|---|
| II-5 | 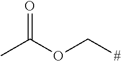 | 1H-NMR(DMSO-d6) δ :2.04(s, 3H), 2.90-3.00(m, 1H), 3.44-3.50(m, 2H), 3.64-3.72(m, 1H), 3.95-4.00(m, 1H), 4.11-4.10(m, 1H), 4.20- 4.30(m, 2H), 5.40-5.5.46(m, 1H), 6.62-5.75(m, 4H), 6.80-6.90(m, 1H), 6.98-7.10(m, 1H), 7.11-7.20(m, 2H), 7.21-7.30(m, 1H), 7.45-7.50(m, 2H) | |
| II-7 | 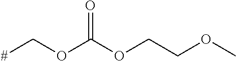 | 1H-NMR(CDCl3) δ :2.85-2.97 (m, 1H), 3.38 (s, 3H), 3.39-3.48 (m, 1H), 3.54 (t, J = 10.4Hz, 1H), 3.68 (t, J = 4.4Hz, 2H), 3.74 (dd, J = 2.8Hz, 12.0Hz, 1H), 3.92 (dd, J = 2.8Hz, 10.8Hz, 1H), 4.05 (d, J = 13.6Hz, 1H), 4.36 (q, J = 4.4 Hz, 2H), 4.51 (dd, J = 2.8Hz, 9.6Hz, | |
| | | 1H), 4.65 (d, J = 12.0Hz, 1H), 5.27 (dd, J = 2.0Hz, 13.6Hz, 1H), 5.34 | |
| | | (s, 1H), 5.86 (d, J = 8.0Hz, 1H), 5.93 (s, 2H), 6.81-6.89 (m, 2H), | |
| | | 6.98-7.16 (m, 5H). | |
| II-8 | 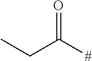 | 1H-NMR (CDCl3) δ: 1.33 (3H, t, J = 7.0 Hz), 2.82 (2H, d, J = 6.1 Hz), 2.93 (1H, t, J = 11.2 Hz), 3.42 (1H, t, J = 11.4 Hz), 3.59 (1H, t, J = 10.2 Hz), 3.78 (1H, d, J = 11.2 Hz), 3.96 (1H, d, J = 10.3 Hz), 4.06 (1H, d, J = 13.8 Hz), 4.55 (1H, d, J = 8.9 Hz), 4.63 (1H, d, J = 13.6 Hz), 5.29 (1H, d, J = 13.9 Hz), 5.36 (1H, s), 5.88 (1H, d, J = 7.4 Hz), 6.90 (1H, s), 7.03-7.12 (6H, m). | |
| II-9 | 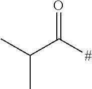 | 1H-NMR (CDCl3) δ: 1.42 (d, J = 6.8 Hz, 6H), 2.85-3.05 (m, 2H), 3.40-3.49 (m, 1H), 3.59 (t, J = 10.4 Hz, 1H), 3.76 (d, J = 11.4 Hz, 1H), 3.94 (d, J = 10.4 Hz, 1H), 4.06 (d, J = 14.1 Hz, 1H), 4.51-4.57 (m, 1H), 4.59-4.70 (m, 1H), 5.25-5.32 (m, 1H), 5.35-5.39 (m, 1H), 5.80-5.89 (m, 1H), 6.85-7.15 (m, 7H). | |
| II-10 | 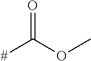 | LC/MS (ESI):m/z = 542 [M + H]+, RT = 1.92 min, method (1) | |
| II-11 | 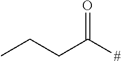 | LC/MS (ESI):m/z = 554 [M + H]+, RT = 2.10 min, method (1) | |

In one example, the neuraminidase inhibitor is one or more of oseltamivir, zanamivir and peramivir. In one example, the neuraminidase inhibitor is oseltamivir.

In one example, at least one systemic symptom is present and includes one or more of headache, feverishness, chills, muscular pain, joint pain, and fatigue.

In one example, at least one respiratory symptom is present and includes one or more of coughing, sore throat, and nasal congestion.

In one example, the severe influenza condition can include one or more of (a) being hospitalized due to the influenza virus infection, (b) requiring an extension of hospitalization because of acquiring the influenza virus infection during the hospitalization, (c) having a National Early Warning Score 2 of four or more, (d) being on support for respiration, and (e) having at least one complication attributable to the influenza virus infection that necessitates hospitalization.

In one example, the severe influenza condition is a National Early Warning Score 2 (NEWS2) of four or more. The meaning of NEWS2 is described as follows.

The original NEWS was created in the United Kingdom by the Royal College of Physicians in 2012 to standardize the process of recording, scoring, and responding to changes in routinely measured physiological parameters in acutely ill patients. The score has been widely implemented across the National Health Service in the United Kingdom, and in other healthcare settings across the world. NEWS2 is a similar but updated score to incorporate an additional oxygen scale for patients at risk of hypercapnic respiratory failure (target oxygen saturation of 88%-92%, rather than the standard target of ≤96%). See Royal College of Physicians, National early warning score (NEWS) 2 Standardising the assessment of acute-illness severity in the NHS. London: RCP, 2017.

The following sevenphysiological parameters are routinely recorded:
Respiration rate
Oxygen saturation
Supplemental oxygen
Systolic blood pressure
Pulse rate
Level of consciousness and new confusion (ACVPU: A=alert, C=new confusion, V=responsive to voice, P=responsive to pain, U=unconscious)
Temperature

In addition, a weighting score of 2 is added for patients requiring supplemental oxygen to maintain their prescribed oxygen saturation range.

NEWS2 was developed for patients aged 2-16 years; however, children aged 12-16 years have very similar physiological parameter ranges as adults. In the present invention, NEWS2 is not used as an early warning system to identify patients who may need escalating levels of care; rather, it is used to standardize vital sign collection which will facilitate setting severity levels for inclusion and demonstrating response to treatment. Most pediatric early warning scores are complex and designed to detect critical illness in younger children. In the present invention, a patient achieving a NEWS2 of greater than or equal to 2 represents a degree of clinical improvement (i.e., clinically stable and potentially eligible for discharge). The time to clinical improvement is defined as the time to hospital discharge or time to NEWS2 of 2 and maintained for 24 hours, whichever comes first. Importantly as well, the method of statistical analysis and the selection of a clinically meaningful difference between treatment groups are possible for an endpoint of this design.

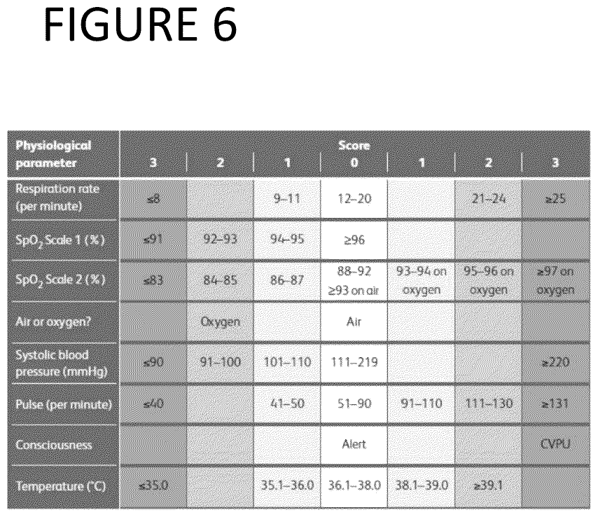

A summary of the NEWS2 scoring system is provided in the table in FIG. 6. An explanation of the table is provided as follows.

At screening, the NEWS2 should be calculated based upon vital sign values recorded during the patient assessment.

The oxygen saturation should be scored according to either the SpO₂ Scale 1 or 2 presented in the table. The SpO₂ Scale 2 is for patients with a target oxygen saturation requirement of 88%-92% (e.g., in patients with hypercapnic respiratory failure related to advanced lung diseases, such as chronic obstructive pulmonary disease (COPD]). This should only be used in patients confirmed to have hypercapnic respiratory failure by blood gas analysis on either a prior or their current hospital admission.

The decision to use the SpO₂ Scale 2 should be made by the treating physician and should be recorded in the eCRF. In all other circumstances, the SpO₂ Scale 1 should be used.

For physiological parameter “Air or Oxygen?”: any patients requiring the use of oxygen or other forms of ventilation to maintain oxygen saturations and support respiration should be assigned a score of 2.

The consciousness level should be recorded according to the best clinical condition of the patient during the assessment. Patients who are assessed as “Alert” (A) should be assigned a score of 0. Patients assessed as “New Confusion” (C), “Responsive to Voice” (V), “Responsive to Pain” (P), or “Unconscious” should be assigned a score of 3.

Scores should be assigned for respiratory rate, systolic blood pressure, pulse, and temperature according to the table in FIG. 6.

The NEWS2 should be recorded in the eCRF at the screening visit to ensure patients meet the eligibility criteria. In addition to the total NEWS2, the individual components of the score should also be recorded in the eCRF. Additional NEWS2 values will be calculated electronically throughout the study by the sponsor based upon entry of vital sign parameters by the investigator in the appropriate eCRF.

### Example Case Calculation:

An 82-old woman was admitted to an acute medical unit from a residential care home. Her observed physiological parameters and corresponding NEWS2 score were as follows:

TABLE 2

| | | Component |
|---|---|---|
| Physiological Parameter | Observation | Score |
| Respiratory rate (per min) | 26 | 3 |
| Oxygen saturations | 95% | 1 |
| (SpO2%) | | |
| Supplemental Oxygen | No | 0 |
| Systolic blood pressure | 140 | 0 |
| (mmHg) | | |
| Pulse Rate (bpm) | 109 | 1 |
| Conscious level | Alert | 0 |
| Temperature (° C.) | 39 | 1 |
| | Total NEWS2 | 6 |
| | Score | |

In one example, the severe influenza condition is the need for support for respiration. In one example, the support for respiration is at least one of a ventilator and an inhalation of oxygen from non-atmospheric oxygen supply, and an oxygen concentrator which concentrates the atmospheric oxygen.

In one example, at least one complication attributable to the influenza virus infection is present. In one example, the complication attributable to the influenza virus infection is one or more of inflammation of heart, brain, or muscle tissues, and muti-organ failure. In one example, the complication attributable to the influenza virus infection is one or more of pneumonia, central nervous system involvement, myositis, rhabdomyolysis, encephalitis, encephalopathy, severe dehydration myocarditis, pericarditis, otitis media, sinusitis, exacerbation of ischemic heart disease, sepsis, acute lung injury, or acute respiratory distress syndrome, and acute exacerbation of chronic kidney disease or respiratory diseases, for example asthma or chronic obstructive pulmonary disease.

In one example, the effective amount of the compound (A) is in a range from about 0.1 mg to about 240 mg. In another example, the effective amount of the compound (A) is in a range from about 3 mg to about 80 mg per dose.

In one example, the effective amount of the compound (A) is adminstered two times or three times in total.

In one example, the compound (A) can be administered as a weight-based dose. In one example, at or about 40 mg is administered to a subject weighing about 40 kg to less than about 80 kg. In one example, about 80 mg is administered to a subject weighing at least about 80 kg. In one example, the compound (A) is administered on the first day of administration upon onset of at least one symptom of an influenza virus infection and again three days later.

In one example, the compound (A) is further administered six days after the first day of administration upon onset of at least one symptom of an influenza virus infection. In one example, the compound (A) is administered at six days after the first day of administration upon onset of at least one symptom of an influenza virus infection in the case that the subject does not show an improvement in at least one condition selected from the group consisting of (i) continuous use of a ventilator, (ii) continuous fever, (iii) severe immune deficiency, and (iv) any complication attributable to the influenza virus infection, as compared to the respective conditions of a subject that has been administered with none of anti-influenza drug including the compound (A) and the compound (B).

In one example, the subject is a patient that is younger than 1 year. In this instance:
(a) if the patient is younger than 3 months, then the effective amount is 0.8-1.2 mg/kg body weight, preferably about 1 mg/kg body weight;
(b) if the patient is 3 months or older but younger than 12 months, then the effective amount is 1.8-2.2 mg/kg body weight, preferably about 2 mg/kg body weight;

In one example, the subject is a patient that is 1 year or older but younger than 12 years. In this instance:
(a) if the patient has a body weight of less than 20 kg, then the effective amount is 1.8-2.2 mg/kg body weight, preferably about 2 mg/kg body weight; or
(b) if the patient has a body weight of 20 kg or more, then the effective amount is 35-45 mg, preferably about 40 mg.

In one example, the compound (A) is administered at or before about 120 hours after the onset of the disease in the subject. In one example, the compound is administered at or before about 72 hours, alternately at or before about 84 hours, alternately at or before about 96 hours, alternately at or before about 120 hours, alternately at or before about 144 hours, alternately at or before about 168 hours, after the onset of the disease in the subject. In one example, the compound is administered at or before about 96 hours after the onset of the disease in the subject. In another example, the compound is administered at or before about 84 hours after the onset of the disease in the subject.

In one example, the amount of the compound administered is effective such that a reduction in a time to show clinical improvement in the subject is statistically significant as compared to that of a non-treated subject. In one example, a non-treated subject is a subject that has not been treated with the compound (A) and the compound (B). In one example, the time to clinical improvement is a time until hospital discharge or a time until a National Early Warning Score 2 of two or less is maintained for at least 24 hours.

In one example, a reduction in the time to show clinical improvement in a subject is statistically significant relative to that of a non-treated subject, where a p-value indicating the statistical significance is less than 0.05, alternately 0.03 or less, alternately 0.02 or less, alternately 0.003 or less, alternately 0.001 or less, alternately 0.001 or less.

In one example, the amount of the compound administered is effective such that a reduction in a time to show clinical improvement in the subject is statistically significant as compared to that of a subject treated with the compound (B). In one example, a p-value indicating the statistical significance of the reduction in a time to show clinical improvement relative to that of a subject treated with the compound (B) is less than 0.05, alternately 0.03 or less, alternately 0.02 or less, alternately 0.003 or less, alternately 0.001 or less, alternately 0.001 or less.

In one example, the compound (B) is administered only once. In one example, the compound (B) is administered daily for up to five days after the onset of the influenza virus infection. In one example, the compound (B) is administed once daily for up to ten days after the onset of the influenza virus infection.

In one example, the effective amount of the compound (B) is in a range from about 0.1 to about 6000 mg as an active compound. In another example, the effective amount of the compound (B) is in a range from about 0.1 to about 1500 mg as an active compound.

In one example, the compound (B) is oseltamivir phosphate, and the effective amount administered is about 75 mg twice daily for five days as an active compound. In another example, the compound (B) is oseltamivir phosphate, and the effective amount administered is about 75 mg twice daily for ten days as an active compound.

In one example, the compound (B) is zanamivir hydrate, and the effective amount administered is 10 mg twice daily for five days as an active compound. In another example, the compound (B) is zanamivir hydrate, and the effective amount administered is 10 mg twice daily for ten days as an active compound.

In one example, the compound (B) is peramivir hydrate. For adults, the effective amount administered is 600 mg once daily for five days as an active compound. For adolescents, the effective amount is 10 mg/kg up to a maximum of 600 mg, once daily for five days as an active compound. In another example, the compound (B) is peramivir hydrate. For adults, the effective amount administered is 600 mg once daily for ten days as an active compound. For adolescents, the effective amount is 10 mg/kg up to a maximum of 600 mg, once daily for ten days as an active compound.

In one example, the compound (A) and the compound (B) are administered orally. In another example, the compound (A) and the compound (B) are administered parenterally. In one example, the compound (A) and the compound (B) are administered through at least one route selected from the group consisting of orally, dermally, subcutaneously, intravenously, intraarterially, intramuscularly, intraperitoneally, transmucosally, via inhalation, transnasally, ophthalmically, via an inner ear and vaginally.

Generally, the compound (A) and the compound (B) can be administered with any material in any amounts that are suitable for use with the compound. In one example, the compound (A) and the compound (B) are administered in combination with at least one material selected from the group consisting of an RNA-dependent RNA polymerase inhibitor, an M2 protein inhibitor, a PB2 Cap binding inhibitor, a HA maturation inhibitor, a recombinant sialidase, a re-assemble inhibitor, RNA interference compound, a receptor of hemagglutinin binding inhibitor, a membrane of HA fusion inhibitor, a NP nuclear translocation inhibitor, a CXCR inhibitor, a CRM1 inhibitor, an anti-HA antibody and an immunological agent.

In one example, the compound (A) and the compound (B) are administered in combination with one or more of laninamivir, favipiravir, amantazine, flumazine,

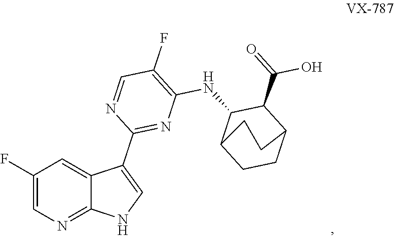 [SMILES: O=C(O)[C@H]1C2CCC(CC2)[C@@H]1Nc1nc(-c2c[nH]c3ncc(F)cc23)ncc1F]

MHAA4549A (as described in McBride et al., Antimicrobial Agents and Chemistry, Vol. 61, Issue 11, (2017)), TCN-032 (as described in Ramos et al., JID 2015:11 (2015)), VIS-410 (as described in Tharakaraman et al., PNAS, vol. 112, no. 35, 10890-10895 (2015)), CR-8020 (as described in Ekiert et al., Science, 333(6044), 843-850 (2011)), CR-6261 (as described in Ekiert et al., Science, 324(5924), 246-251 (2009)), CT-P27 (as described in Celltrion, Press Release, Oct. 12, 2016) and MEDI-8852 (as described in Cell, 166(3), 596-608 (2016)).

In one example, the compound (A) and the compound (B) are administered in at least one form selected from the group consisting of a tablet, powder, a granule, a capsule, a pill, a film, a suspension, an emulsion, an elixir, a syrup, lemonade, spirit, aromatic water, extract, decoction and tincture.

In one example, the compound (A) and the compound (B) are administered in at least one form selected from the group consisting of a sugar-coated tablet, a film-coated tablet, an enteric-coated tablet, a sustained-release tablet, a troche tablet, a sublingual tablet, a buccal tablet, a chewable tablet, an orally disintegrated tablet, a dry syrup, a soft capsule, a micro capsule or a sustained-release capsule.

In one example, the compound (A) and the compound (B) are administered in at least one form selected from the group consisting of an injection, an infusion, an eye drop, a nose drop, an ear drop, an aerosol, an inhalation, a lotion, an impregnation, a liniment, a mouthwash, an enema, an ointment, a plaster, a jelly, a cream, a patch, a cataplasm, an external powder or a suppository.

### Test Example 1: BA Test

Materials and methods for experiments to evaluate oral absorption
(1) Experimental animals: mice or SD rats were used.
(2) Rearing condition: mice or SD rats were fasted and were allowed free access to sterilized tap water.
(3) Setting of dosage and grouping: Oral administration and intravenous administration were performed with the predetermined dosage. Grouping was set as below. (Dosage was changed per compound)
   Oral administration 1 to 30 mg/kg (n=2 to 3)
   Intravenous administration 0.5 to 10 mg/kg (n=2 to 3)
(4) Preparation of administration solutions: Compounds (II-4), (II-5), (II-6), (II-7), (II-8), (II-9), (II-10), and (II-11) were prepared for evaluation in rats. Oral administration was performed as solution or suspension. Intravenous administration was performed after solubilization.
(5) Routes of administration: Oral administration was performed mandatory into the stomach by oral sonde. Intravenous administration was performed from caudal vein by syringes with needle.
(6) Evaluation items: Blood was collected serially and concentration of a compound used in the present invention in plasma was measured by LC/MS/MS.
(7) Statistical analysis: About transition of concentration of a compound used in the present invention in plasma, the area under the plasma concentration versus time curve (AUC) was calculated by non-linear least-squares method program, WinNonlin (a registered trademark), and bioavailability (BA) of a compound used in the present invention was calculated from AUCs of the oral administration group and the intravenous administration group. The BAs of each compound are described in Table 3 below.

### (Result)

TABLE 3

| No. | BA (%) |
|---|---|
| II-4 | 20.0 |
| II-5 | 17.8 |
| II-6 | 14.9 |
| II-7 | 14.5 |
| II-8 | 27.8 |
| II-9 | 15.0 |
| II-10 | 10.6 |
| II-11 | 11.0 |
| III | 4.2 |

Based on the above results, all of the prodrug compounds (Compound (II)) had improved bioavailability compared to Compound (III).

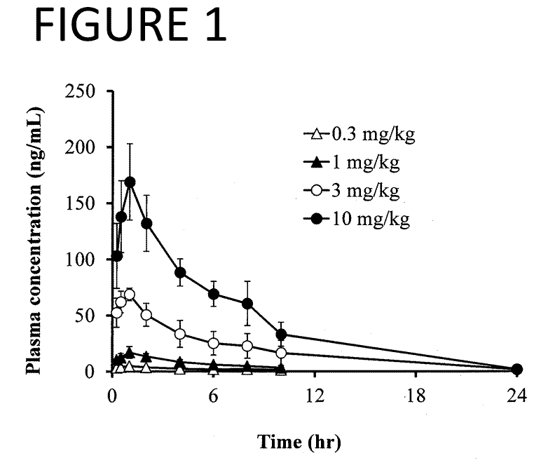

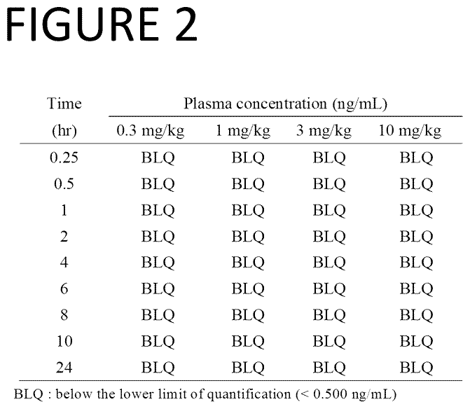

FIGS. 1 and 2 show a result of measuring the plasma concentration of Compound (III) and Compound (II-6), respectively, after oral administration of prodrug Compound (II-6) to rats under non-fasting conditions.

As shown in FIG. 2, the concentration of Compound (II-6) in all plasma samples was below the limit of quantification (<0.500 ng/mL) for all time points tested (0.25 h, 0.5 h, 1 h, 2 h, 4 h, 6 h, 8 h, 10 h, and 24 h) and for all doses tested (0.3 mg/kg, 1 mg/kg, 3 mg/kg, and 10 mg/kg). Therefore, prodrug Compound (II-6) was found to have metabolized promptly to Compound (III) in vivo after administration.

Based on the above test results, it was revealed that the prodrug compounds (Compound (II)) were absorbed into the body after oral administration, and rapidly converted into Compound (III) in the blood. The prodrug compounds used in the present example also showed excellent oral absorbability. Therefore, the prodrug compounds used in the present example, including Compound (II-6), can be useful agents for treatment and/or prevention of symptom and/or disease induced by infection with influenza virus.

### Test Example 2: Bacterial Co-Infection Mouse Model for Delayed Treatment with BXA

### (1) Materials and Methods

### (1.1) Compounds

BXA, the active form of BXM, was synthesized at Shionogi & Co., Ltd. (Osaka, Japan) (Shionogi). Oseltamivir phosphate (“OSP”) was purchased from AK Scientific, Inc. (Union City, CA, USA). Suspension of BXA and solution of OSP were prepared with 0.5% methylcellulose 400 solution (MC, FUJIFILM Wako Pure Chemical Corporation, Osaka, Japan). Administration dose was 0.2 mL per mouse. Doses of BXA and OSP were selected based on human doses in clinics. In this study, suspension of BXA was subcutaneously administered in mice to maintain the plasma concentration of BXA as seen in humans.

### (1.2) Virus and Bacteria

A/Osaka/129/2009 strain of influenza virus and Streptococcus pneumoniae SR1326 strain of bacteria were clinically isolated in Japan individually and adapted to mice in Shionogi.

### (1.3) Animals

Specific-pathogen-free 6-week-old female BALB/c mice (Charles River Laboratories Japan, Inc.) were used in the study. Body weights and survival were monitored once daily. The mice were immediately euthanized and regarded to be dead in the analysis for survival time when they lost more than 30% of their body weight compared to their weight pre-infection of virus according to humane endpoints. Upon virus or bacteria inoculation, mice were anesthetized by intramuscular administration at 100 μL of anesthetic solution containing 0.03 mg/mL medetomidine hydrochloride, 0.4 mg/mL midazolam, 0.5 mg/mL butorphanol tartrate in saline. All mouse studies were conducted under applicable laws and guidelines and with the approval of the Shionogi Animal Care and Use Committee (Approval number: S18060D-0002).

### (2) Antiviral Study in Mouse Models

### (2.1) Effect of 2-Day Delayed Administration of BXA in a Lethal Co-Infection Mouse Model

Mice were inoculated with 100 μL of A/Osaka/129/2009 (mouse-adapted, 1.00×10³ TCID₅₀) under anesthesia. At 2 days after virus infection, the mice were intranasally inoculated with 100 μL of Streptococcus pneumoniae SR1326 (mouse-adapted, 1.00×10² CFU) under anesthesia. Starting on day 2 (2 days after virus infection), the mice (n=4 to 5 per group) were treated subcutaneously with BXA at a dose of 10 mg/kg (single dose) or orally with OSP at a dose of 10 mg/kg (bid for 5 days). To examine the effect of combination treatment with these substances, mice were treated with both BXA subcutaneously at a dose of 10 mg/kg (single dose) and OSP orally at a dose of 10 mg/kg (bid for 5 days). Control mice were treated subcutaneously with 0.5% MC (single dose). Mice were examined once daily for survival and body weight through 15 days post virus infection.

### (2.2) Effect of 3- or 4-Day Delayed Administration of BXA in a Lethal Infection Mouse Model

Mice were inoculated with 100 μL of A/Osaka/129/2009 (mouse-adapted, 1.00×10² TCID₅₀) under anesthesia. At 2 days after virus infection, the mice were intranasally inoculated with 100 μL of Streptococcus pneumoniae SR1326 (mouse-adapted, 1.00×102 CFU) under anesthesia. Starting on day 3 or 4 (3 or 4 days after virus infection), the mice (n=5 per group) were treated subcutaneously with BXA at a dose of 10 mg/kg (single dose) or orally with OSP at a dose of 10 mg/kg (bid for 5 days). To examine the effect of combination treatment with these substances, mice were treated with both BXA subcutaneously at a dose of 10 mg/kg (single dose) and OSP orally at a dose of 10 mg/kg (bid for 5 days). Control mice were treated subcutaneously with 0.5% MC (single dose). Mice were examined once daily for survival and body weight through 15 days post virus infection.

### (3) Statistical Analysis

Differences in survival time after virus infection were analyzed by log rank test. Statistical analysis was performed using the statistical analysis software, SAS version 9.2 for Windows (SAS Institute, Cary, NC). Two-sided adjusted P values below 0.05 were considered as statistically significant.

### (4) Results

### (4.1) Effect of 2-Day Delayed Administration of BXA in a Lethal Co-Infection Mouse Model

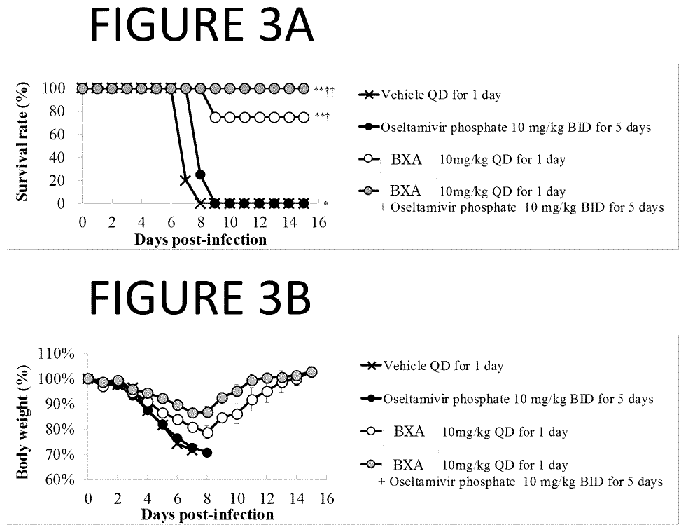

The effects of 2-day delayed administration of BXA were examined in mice lethally co infected with influenza type A virus and Streptococcus pneumoniae. All vehicle-treated mice inoculated with A/Osaka/129/2009 (mouse-adapted, 1.00×10³ TCID₅₀) and Streptococcus pneumoniae SR1326 (mouse-adapted, 1.00×10² CFU) died by day 8 post virus infection. When treatment delayed until 2 days post infection, 75% of mice treated with 10 mg/kg of BXA survived, whereas all mice died in OSP-treated group (FIG. 3A). As a result, treatment with BXA significantly prolonged survival time compared with vehicle treatment. Comparing the efficacy of BXA and OSP on survival time, the survival time in the group given BXA was significantly prolonged compared with that of the OSP-treated group. The combination treatment of BXA and OSP provided more protection against mortality than either monotherapy alone. To further characterize the effects of delayed treatment with BXM, we compared body weight change after virus infection. In the group treated with BXM showed less reduction of body weight compared with the vehicle- or OSP-treated group (FIG. 3B). Combination treatment with BXM+OSP was more effective than either monotherapy in ameliorating the loss of body weight.

### (4.2) Effect of 3- or 4-Day Delayed Administration of BXA in a Lethal Infection Mouse Model

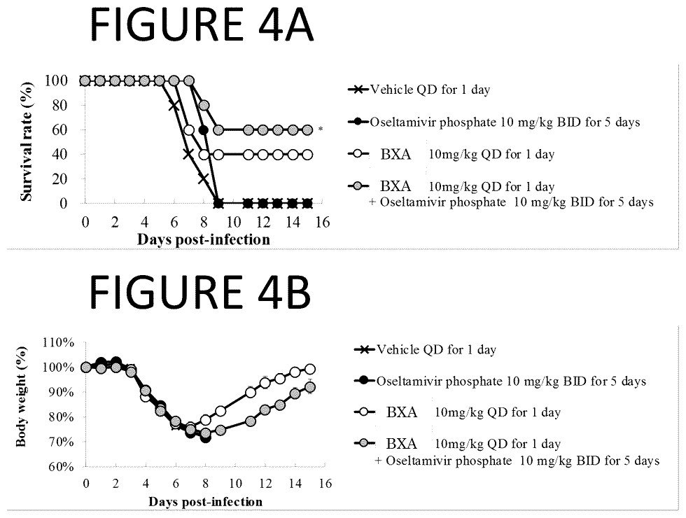

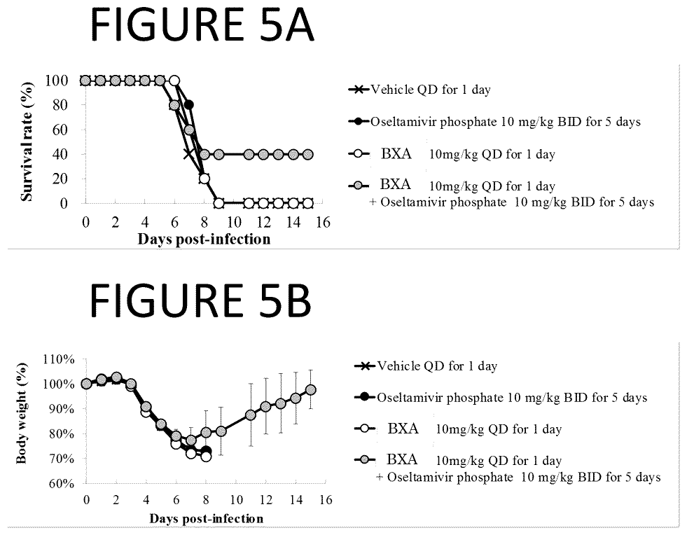

Next, the effects of 3- or 4-day delayed administration of BXA were examined in mice lethally co infected with influenza type A virus and Streptococcus pneumoniae. All vehicle-treated mice inoculated with A/Osaka/129/2009 (mouse-adapted, 1.00×10² TCID₅₀) and Streptococcus pneumoniae SR1326 (mouse-adapted, 1.00×10² CFU) died by day 9 post infection. When treatment delayed until 3 days post virus infection, 40% and 60% of mice recovered from body weight loss before reaching the humane endpoints and survived in the groups treated with BXA alone and combination of BXA+OSP, respectively (FIGS. 4A and 4B). As a result, combination treatment with BXA+OSP significantly prolonged survival time compared with vehicle treatment. Even when combination treatment of BXA+OSP was initiated at 4 days post virus infection, 40% of mice recovered from body weight loss before reaching the humane endpoints and survived, whereas all mice died in either monotherapy group (FIGS. 5A and 5B).

Both epidemic and pandemic influenza virus infections are major public health concerns, but, no antiviral drug has been shown to definitively reduce hospitalization or mortality in a randomized clinical trial. Therefore, novel anti-influenza drugs that offer significant improvement over current therapy are urgently needed. In serious cases in clinic, co-infection of bacteria (ex Streptococcus pneumoniae) following influenza virus infection is considered to be one of the crucial causes of severe pneumoniae or death (Metersky et al., International Journal of Infectious Diseases 16 (2012) e321-e331), suggesting that the bacterial co-infection mouse model may be useful to predict the efficacy of BXM in serious cases. In this nonclinical study, we evaluated the therapeutic efficacy of BXA (the active form of BXM) comparing that of OSP in a bacterial co-infection mouse model. We have previously confirmed that the time-course change of plasma concentration mimicked human PK curve in mice administered subcutaneously with BXA compared to that in mice administered with BXM orally. Therefore, in this study, suspension of BXA (10 mg/kg) was subcutaneously administered in mice to maintain the plasma concentration of BXA as seen in humans. The dose of OSP was calculated based on human pharmacokinetic data: in mice, 5 mg/kg twice daily is equivalent to the human clinical dose, and for the treatment of critically ill patients double the high dose is recommended. Therefore, in this study, mice were treated with 10 mg/kg twice daily for 5 days (double the high dose) of OSP as the reference drug treatment (Ward et al., J. Antimicrob Chemother., February:55 Suppl 1: 15-121 (2005)).

Bacterial co-infection occurs shortly after the viral infection or during the period of viral shedding in human (Chertow et al., JAMA. 2013; 309(3):275-282.). We have previously confirmed that the virus load reaches peak levels within 2 days after infection of A/Osaka/129/2009 (mouse-adapted) to mice (Onishi et al., Antiviral Research 117 (2015) 52-59). Therefore, in this study, the mice were inoculated with Streptococcus pneumoniae at 2 days after virus infection. In our model, a sign of co-infection appears initially as the loss of body weight at day 3 after virus infection. Therefore, we evaluated the effect of delayed treatment up to 4 days after virus infection, considering that antiviral therapy may be started after onset of influenza symptoms in clinic. When the treatment with BXA was delayed up to 2 or 3 days after infection, BXA was more effective for the prevention of mortality than administration of vehicle or OSP in mice infected with influenza type A virus followed by Streptococcus pneumoniae. In contrast, when treatment with OSP began at 2 or 3 days after virus infection, all mice died within 9 days. Although either monotherapy of BXA or OSP was not effective when the treatment delayed 4 days after virus treatment, survival rate was improved by combination treatment of BXA+OSP compared to vehicle treatment. These findings support BXM and its combination therapy with OSP as treatment options for patients with co-infection of influenza virus and Streptococcus pneumoniae.

### Test Example 3: Clinical Trial

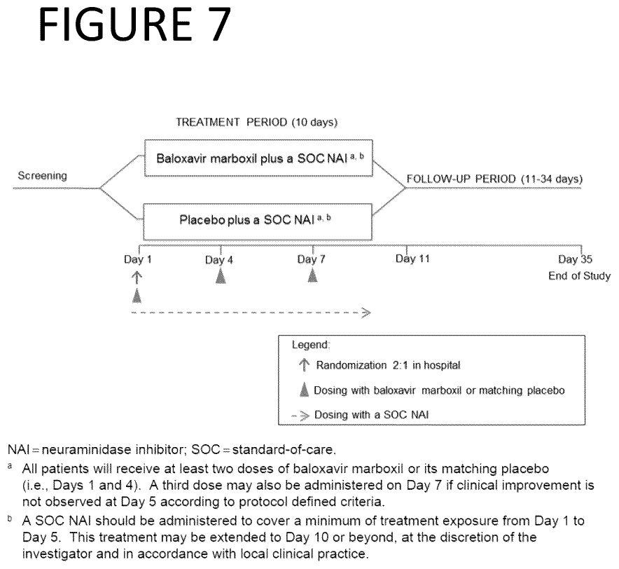

A randomized, double-blind, placebo-controlled, multicenter study was conducted to assess the efficacy, safety, and pharmacokinetics of baloxavir marboxil in combination with a standard of care neuraminidase inhibitor (“SOC NAI”) (i.e., oseltamivir, zanamivir, or peramivir), compared with a matching placebo in combination with a SOC NAI in approximately 240 hospitalized adults and adolescent patients (aged ≥12 years) with influenza virus infection. FIG. 7 presents an overview of the study design.

Patients were randomized as soon as possible after screening, providing they were within 96 hours of symptom onset and hospitalized (includes assessment in emergency centers pending admission to a hospital ward). Patients were assigned in a 2:1 ratio to receive baloxavir marboxil or matching placebo. Study treatment was given in combination with a SOC NAI according to investigator preference (i.e., oseltamivir, zanamivir, or peramivir).

Baloxavir marboxil was administered as a weight-based dose (40 mg for patients weighing 40 to <80 kg, 80 mg for patients weighing ≥80 kg) on Days 1 and 4, and also on Day 7 if clinical improvement had not occurred at Day 5. Clinical improvement is evaluated based on (i) continuous use of a ventilator, (ii) continuous fever, (iii) severe immune deficiency and/or (iv) any complication attributable to the influenza virus infection. Specific criteria for extended dosing to Day 7 include ongoing mechanical ventilation, persistence of fever, severely immunocompromised, confirmed/suspected influenza virus infection-related complication, or pneumonia.

Seriously ill patients who are hospitalized with influenza virus infection demonstrate prolonged viral shedding compared with otherwise healthy patients with influenza virus infection. Therefore, the repeat-dose regimen was to ensure that plasma baloxavir concentrations remain above a target-threshold concentration for a longer duration in severely ill patients owing to the greater potential of a protracted influenza illness.

Patients were administered up to three doses of baloxavir marboxil separated by 3 days (i.e., Days 1, 4, and 7). The dosing interval of 3 days approximates terminal disposition half-life (˜70 hours) of baloxavir, thus higher drug exposure on Day 4 or 7 relative to the first dose was expected, under the assumption of linear pharmacokinetics. Model-based simulation indicates that the population-median accumulation in terms of maxim um plasma concentration (Cₘₐₓ) and minimum concentration (C_{trough}) prior to next dose on Day 7 (third consecutive dose) would be approximately 1.5 and 1.7 times greater than after the first dose, respectively. Based on the worst-case arithmetic mean Bayesian estimate of 126 ng/ml for Cₘₐₓ in Phase III (Asian >80 kg), the highest peak plasma concentration after a third consecutive dose (Day 7) was not expected to exceed the peak drug levels observed in Japanese patients receiving 80 mg in the Thorough QT (TQT) study. Likewise, total drug exposure simulated on Day 7 area under the plasma concentration-time curve for a dosing interval (AUCₜₐᵤ) was not expected to exceed area under the concentration-time curve from Time 0 to infinity (AUC_{inf}) of the TQT, regardless of dose. However, for certain individual Asian patients receiving 80 mg, total drug exposure on Day 7 (Aₜₐᵤ) may approach similar overall exposures as seen in the TQT.

Treatment with a SOC NAI (i.e., oseltamivir, zanamivir, or peramivir) was administered in accordance with local clinical practice. However, due to the design of the trial, wherever possible, treatment with the chosen SOC NAI was made sufficient to ensure exposure of active drug such that anti-viral activity was maintained from Day 1 through Day 5. Table 4 provides details on the choice of dose and regimen.

TABLE 4

| | | | Duration | |
|---|---|---|---|---|
| | | | of | |
| | | | Dosing | |
| | | | from | |
| NAI | Dose | Regimen | Day 1 | Comments |
| Oseltamivir | 75 mg | BID | 5 days | |
| Zanamivir | 10 mg | BID | 5 days | Lowest age for |
| | | | | dosing to be in |
| | | | | line with local |
| | | | | prescribing |
| | | | | information |
| Peramivir | Adults: | QD | 5 days | Peramivir dosing |
| | 600 mg | | | regimen has been |
| | Adolescents: | | | selected in line with |
| | 10 mg/kg | | | the study conducted |
| | up to, a | | | in hospitalized |
| | maximum | | | patients |
| | of | | | (deJong et al. 2014) |
| | 600 mg. | | | and is, therefore, |
| | | | | higher than the |
| | | | | generally approved |
| | | | | dose in |
| | | | | uncomplicated |
| | | | | influenza |

BID = twice daily; NAI = neuraminidase inhibitor; QD = once daily.

The SOC NAI treatment was extended to Day 10 or beyond at the discretion of the investigator and in accordance with local clinical practice.

The study consisted of two periods: a 10-day treatment period and a 25-day follow-up period. Therefore, the maximum study duration for each patient was 35 days.

Generally, two measures of clinical improvement were used in this study: “Time to clinical improvement” as the primary endpoint and “Response rates of the 6-point ordinal scale” in an alpha-protected hierarchy as the key secondary endpoint. Finally, as an exploratory endpoint, “Time to clinical response” as defined in a previous zanamivir clinical study in this patient population also was evaluated.

The primary efficacy objective for this study was to evaluate the efficacy of baloxavir marboxil plus a SOC NAI, compared with matching placebo plus SOC NAI on the basis of the following endpoint:
TTCI (time to clinical improvement, defined as time to hospital discharge or time to NEWS2 of ≤2 and maintained for 24 hours, whichever comes first)

The median TTCI was compared between the baloxavir marboxil plus SOC NAI and matching placebo plus SOC NAI arms using the stratified log-rank test within three regions (i.e., North America; Europe, Middle East, and Africa (EMEA); rest of the world ROW), NEWS2 (≤7, >7), and time from symptom onset to study treatment (≤48 hours, >48 hours) included as the stratification factors. The Kaplan-Meier plot, median time to response, 95% CIs, and p-values were determined.

The log-rank test requires the assumption of proportional hazards to be met. The proportional hazards assumption was tested graphically using the log cumulative hazard plot by treatment group. The plots for each treatment group are parallel if the proportional hazard assumption holds. In the event the proportional hazards assumption was violated the Gehan-Wilcoxon test was used to analyze the data.

The estimand is the median change in TTCI. This absolute measure was assessed over the duration of the study (35 days). Intercurrent events are those that occur after treatment initiation and either preclude observation of the variable or affect its interpretation. Intercurrent events were accounted for through censoring rules. Patients who withdrew, who were lost to follow-up, who did not have a clinical response event, or who died were censored at their last contact date or date of death, whichever was applicable. No dose reductions or treatment cross-overs were anticipated.

The primary efficacy analysis population was characterized through the inclusion/exclusion criteria and was the modified-ITTI population, which consisted of all patients who were randomized to treatment, received a dose of study drug, and were RT-PCR positive for influenza virus infection.

A sensitivity analysis was conducted where patients who were lost to follow-up or who did not have a clinical response event were assumed to have had an event at 28 days. Patients who died were censored at their date of death.

The primary endpoint was the time to clinical improvement (TTCI), defined as time to hospital discharge or time to National Early Warning Score 2 (NEWS2) of 2 maintained for 24 hours, whichever occurs first. This endpoint provides objective evidence of improvement in a patient's condition, which is a reasonable surrogate to overall clinical status improvement.

The key secondary endpoint was the proportion of patients per category in the 6-point ordinal scale at Day 7. The categories within this scale reflect the clinical status of patients during their hospital stay. The clinical status was assessed to reflect the most accurate condition of the patient from a medical perspective, to minimize the effects of administrative or non-medical factors (e.g., the hospital bed availability may lead to patients not being cared for in the ward most suitable for their condition). The 6-point ordinal scale was used in this study to investigate the change in clinical state of patients as the key secondary endpoint.

Assessment of patient status using an ordinal scale was recorded at baseline on Day 1, and again once daily every morning (between 8 am and 12 pm) while hospitalized. The ordinal scale categories are as follows:
1. Discharged (or “ready for discharge”)
2. Non-ICU hospital ward (or “ready for hospital ward”) not requiring supplemental oxygen/non-invasive ventilation
3. Non-ICU hospital ward (or “ready for hospital ward”) requiring supplemental oxygen/non-invasive ventilation
4. ICU without mechanical (invasive) ventilation (or “ready for ICU admission”)
5. Mechanical (invasive) ventilation
6. Death

Patients ready to be discharged (e.g., still hospitalized due to non-medical or administrative reasons) were assigned an ordinal scale of 1. Patients in non-ICU hospital ward that were eligible for ICU care based on clinical presentation but awaiting ICU care were assigned an ordinal scale of 4. Patients in ICU for administrative or non-medical reasons, who were ready for a non-ICU hospital ward, were assigned an ordinal scale of 2 (if not requiring supplemental oxygen/non-invasive ventilation) or 3 (requiring supplemental oxygen/non-invasive ventilation).

Patients who satisfied all of the following criteria were selected as subjects.
Adult patients: Signed informed consent by any patient capable of giving consent, or, where the patient was not capable of giving consent, by his or her legal/authorized representative
Adolescent patients not able to legally consent: written informed consent for study participation is obtained from patient's parents or legal guardian, with assent as appropriate by the patient, depending on the patient's level of understanding and capability to provide assent
Age ≥12 years at the time of signing the Informed Consent Form/Assent Form
Ability to comply with the study protocol, in the investigator's judgment
Patients who are hospitalized for severe influenza virus infection or acquire influenza virus infection during hospitalization, the severity of which requires an extension of hospitalization
Diagnosis of influenza virus infection with virus type A and/or type B by a positive Rapid Influenza Diagnostic Test (RIDT) or reverse transcriptase-polymerase chain reaction (RT-PCR).
   Positive results from local tests are acceptable if conducted within the 24 hours prior to screening. A patient with a negative RIDT may be enrolled if influenza virus infection is suspected based on local surveillance data or if the patient reports contact with a known case of influenza virus infection within the prior 7 days and all other inclusion criteria are met
The time interval between the onset of symptoms and randomization is within 96 hours The onset of symptoms is defined as the time when the patient experiences at least one new general symptom (e.g., headache, feverishness or chills, muscle or joint pain, fatigue), respiratory symptom (e.g., cough, sore throat, nasal congestion), or fever.
A score of ≥4 based on the NEWS2
Patients will require objective criteria of seriousness defined by at least one of the following criteria:
Requires ventilate ion or supplemental oxygen to support respiration
Has at least one complication related to influenza virus infection that requires hospitalization (e.g., pneumonia, CNS involvement, myositis, rhabdomyolysis, acute exacerbation of chronic kidney disease, asthma or chronic obstructive pulmonary disease (COPD), severe dehydration, myocarditis, pericarditis, exacerbation of ischemic heart disease)
For women of childbearing potential: Agreement to remain abstinent (refrain from heterosexual intercourse) or use contraceptive methods with a failure rate of <1% per year during the treatment period and for 28 days after the last dose of study treatment. Hormonal contraceptive methods must be supplemented by a barrier method.

A woman is considered to be of childbearing potential if she is postmenarcheal, has not reached a postmenopausal state (12 continuous months of amenorrhea with no identified cause other than menopause), and has not undergone surgical sterilization (removal of ovaries and/or uterus).

Examples of contraceptive methods with a failure rate of <1% per year include bilateral tubal ligation, male sterilization, hormonal contraceptives that inhibit ovulation, hormone-releasing intrauterine devices, and copper intrauterine devices.

The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.

### Incidence of Side Effects

The number of side-effect episodes and the number of patients with side effect were counted for each administration group.

### FORMULATION EXAMPLE

The following Formulation Examples are only exemplified and not intended to limit the scope of the invention.

### Formulation Example 1: Tablets

The compounds used in the present invention, lactose, and calcium stearate were mixed. The mixture was crushed, granulated and dried to give a suitable size of granules. Next, calcium stearate was added to the granules, and the mixture was compressed and molded to give tablets.

### Formulation Example 2: Capsules

The compounds used in the present invention, lactose, and calcium stearate were mixed uniformly to obtain powder medicines in the form of powders or fine granules. The powder medicines were filled into capsule containers to give capsules.

### Formulation Example 3: Granules

The compounds used in the present invention, lactose and calcium stearate are mixed uniformly and the mixture is compressed and molded. Then, it is crushed, granulated and sieved to give suitable sizes of granules.

### Formulation Example 4: Orally Disintegrated Tablets

The compounds used in the present invention and crystalline cellulose are mixed, granulated and tablets are made to give orally disintegrated tablets.

### Formulation Example 5: Dry Syrups

The compounds used in the present invention and lactose are mixed, crushed, granulated and sieved to give suitable sizes of dry syrups.

### Formulation Example 6: Injections

The compounds used in the present invention and phosphate buffer are mixed to give injection.

### Formulation Example 7: Infusions

The compounds used in the present invention and phosphate buffer are mixed to give injection.

### Formulation Example 8: Inhalations

The compound used in the present invention and lactose are mixed and crushed finely to give inhalations.

### Formulation Example 9: Ointments

The compounds used in the present invention and petrolatum are mixed to give ointments.

### Formulation Example 10: Patches

The compounds used in the present invention and base such as adhesive plaster or the like are mixed to give patches.

What is claimed is:

1. A method for treating an influenza virus infection, comprising:
administering a combination of an effective amount of a compound (A) and an effective amount of a compound (B) to a subject,
wherein the subject:
(1) has an influenza virus infection,
(2) has been symptomatic of the influenza virus infection for no more than 96 hours, and
(3) further has at least one severe influenza condition selected from the group consisting of:
(a) being hospitalized due to the influenza virus infection,
(b) requiring an extension of hospitalization because of acquiring the influenza virus infection during the hospitalization,
(c) having a National Early Warning Score 2 of four or more,
(d) being on support for respiration, and
(e) having at least one complication attributable to the influenza virus infection that necessitates hospitalization,
wherein the compound (A) has one of following formula (1) or formula (2): 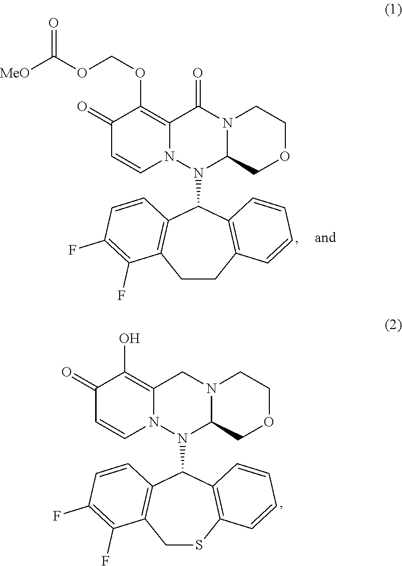
or a pharmaceutically acceptable salt thereof, and
wherein the compound (B) is at least one neuraminidase inhibitor.

2. The method according to claim 1, wherein the at least one neuraminidase inhibitor is at least one compound selected from the group consisting of oseltamivir, zanamivir, peramivir, a pharmaceutically acceptable salt thereof, and a pharmaceutically acceptable solvate thereof.

3. The method according to claim 1, wherein the at least one neuraminidase inhibitor is oseltamivir, a pharmaceutically acceptable salt thereof, or a pharmaceutically acceptable solvate thereof.

4. The method according to claim 1, wherein the subject having been symptomatic of the influenza virus infection has at least one symptom selected from the group consisting of a systemic symptom and a respiratory symptom.

5. The method according to claim 4,
wherein the at least one symptom is the systemic symptom, and
the systemic symptom is at least one symptom selected from the group consisting of headache, feverishness, chills, muscular pain, joint pain, and fatigue.

6. The method according to claim 4,
wherein the at least one symptom is the respiratory symptom, and
the respiratory symptom is at least one symptom selected from the group consisting of coughing, sore throat, and nasal congestion.

7. The method according to claim 1, wherein the severe influenza condition is being on support for respiration, and the support for respiration is selected from the group consisting of a ventilator, inhalation of oxygen from non-atmospheric oxygen supply, and an oxygen concentrator that concentrates atmospheric oxygen.

8. The method according to claim 1, wherein the at least one severe influenza condition is having the at least one complication attributable to the influenza virus infection selected from the group consisting of pneumonia, central nervous system involvement, myositis, rhabdomyolysis, severe dehydration myocarditis, pericarditis, exacerbation of ischemic heart disease, and acute exacerbation of chronic kidney disease, asthma, and chronic obstructive pulmonary disease.

9. The method according to claim 1, wherein the compound (A) is administered to the subject in an amount from about 80 mg to about 240 mg.

10. The method according to claim 1, wherein compound (A) is administered to the subject two times or three times in total.

11. The method according to claim 1, wherein the compound (A) is administered to the subject at day 1 or day 4 after an onset of the influenza virus infection.

12. The method according to claim 1, wherein the compound (A) is further administered to the subject at day 7 after an onset of the influenza virus infection.

13. The method according to claim 1, wherein the effective amount of the compound (B) is in a range from about 0.1 mg to about 6000 mg as an active compound.

14. The method according to claim 1, wherein the compound (B) is administered at one time or daily for up to five days after an onset of the influenza virus infection.

15. The method according to claim 1, wherein the compound (B) is administered at one time or daily for up to ten days after an onset of the influenza virus infection.

16. The method according to claim 1, wherein the amount of the compound (A) and the amount of the compound (B) administered to the subject are effective such that a time to show clinical improvement in the subject is reduced compared to a time to show clinical improvement in a treated subject only with the compound (B).

17. The method according to claim 1, wherein the amount of the compound (A) and the amount of the compound (B) administered to the subject are effective such that a time to show clinical improvement in the subject is reduced compared to a time to show clinical improvement in a non-treated subject.

18. The method according to claim 16, wherein the amount of the compound (A) and the amount of the compound (B) administered to the subject are effective such that the time to show clinical improvement in the subject is statistically significant as compared to the time to show clinical improvement in a treated subject only with the compound (B).

19. The method according to claim 18, wherein a p-value indicating the statistical significance of the time to show the clinical improvement in the subject is less than 0.05.

20. The method according to claim 16, wherein the time to show clinical improvement in the subject is a time to hospital discharge or a time until a National Early Warning Score 2 of two or less is maintained for at least 24 hours.

21. The method according to claim 1, wherein each of the compound (A) and the compound (B) is administered through a route individually selected from the group consisting of orally and parenterally.

22. The method according to claim 1, wherein the compound (A) is administered at day 7 after an onset of the influenza virus infection if the subject does not show an improvement in at least one condition selected from the group consisting of (i) continuous use of a ventilator, (ii) continuous fever, (iii) severe immune deficiency, and (iv) any complication attributable to the influenza virus infection.

23. A method for treating an influenza virus infection, comprising:
reading a dosage instruction on a package insert or in a package of a pharmaceutical formulation comprising a compound (A) having one of following formula (1) or formula (2): 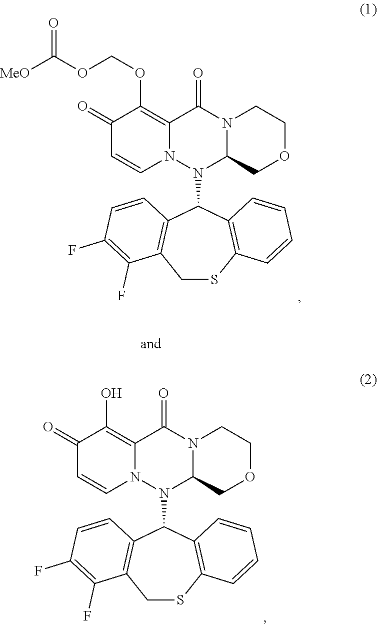 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof, and a compound (B) that is at least one neuraminidase inhibitor; and
administering the pharmaceutical formulation to a subject that:
(1) has an influenza virus infection,
(2) has been symptomatic of the influenza virus infection for no more than 96 hours, and
(3) further has at least one severe influenza condition selected from the group consisting of:
(a) being hospitalized due to the influenza virus infection,
(b) requiring an extension of hospitalization because of acquiring the influenza virus infection during the hospitalization,
(c) having a National Early Warning Score 2 of four or more,
(d) being on support for respiration, and
(e) having at least one complication attributable to the influenza virus infection that necessitates hospitalization.

24. A pharmaceutical composition for treating a subject that:
(1) has an influenza virus infection,
(2) has been symptomatic of the influenza virus infection for no more than 96 hours, and
(3) further has at least one severe influenza condition selected from the group consisting of:
(a) being hospitalized due to influenza virus infection,
(b) requiring an extension of hospitalization because of acquiring the influenza virus infection during the hospitalization,
(c) having a National Early Warning Score 2 of four or more,
(d) being on support for respiration, and
(e) having at least one complication attributable to the influenza virus infection that necessitates hospitalization
wherein the pharmaceutical composition comprises the compound (A) and the compound (B),
the treatment comprises administering an effective amount of a compound (A) and an effective amount of a compound (B) to the subject,
the compound (A) has one of following formula (1) or formula (2): 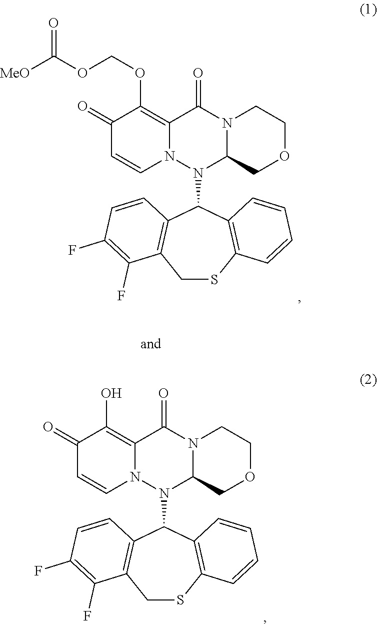 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof, and
the compound (B) is at least one neuraminidase inhibitor.

25. A package, comprising: a pharmaceutical formulation comprising a compound (A) having one of following formula (1) or formula (2): 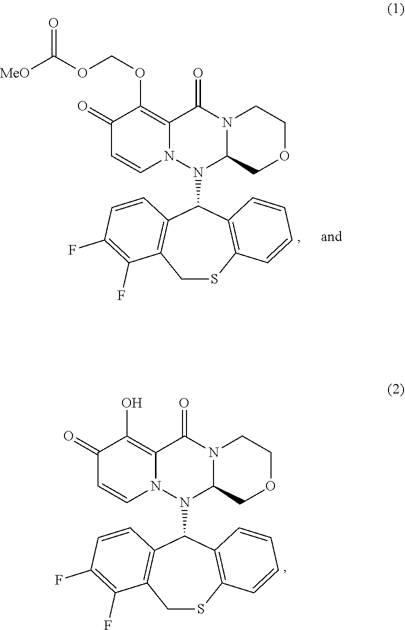 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof, and a compound (B) that is at least one neuraminidase inhibitor; and
a dosage instruction on a package insert or in the package for administering the pharmaceutical formulation to a subject that:
(1) has an influenza virus infection,
(2) has been symptomatic of the influenza virus infection for no more than 96 hours, and
(3) further has at least one severe influenza condition selected from the group consisting of:
(a) being hospitalized due to the influenza virus infection,
(b) requiring an extension of hospitalization because of acquiring the influenza virus infection during the hospitalization,
(c) having a National Early Warning Score 2 of four or more,
(d) being on support for respiration, and
(e) having at least one complication attributable to the influenza virus infection that necessitates hospitalization.
